(19) United States
(12) Patent Application Publication
(10) Pub. No.: US 2025/0179413 A1
(43) Pub. Date: Jun. 5, 2025

(54) METHODS, SYSTEMS AND RELATED MACHINE LEARNING TOOLS FOR CONTROLLED GROWTH OF AERIAL MYCELIUM, INCLUDING TARGETED ASSEMBLY OF MYCELIAL GROWTH SUBSTRATES, SELECTION OF AERIAL MYCELIUM MATERIALS FOR SUITABLE END USES, AND AERIAL MYCELIUM MATERIALS PRODUCED THEREBY

(71) Applicant: Ecovative LLC, Green Island, NY (US)

(72) Inventors: Eben D. Bayer, Troy, NY (US); Peter James Mueller, Poestenkill, NY (US); Julian Noah Rosenberg, Altamont, NY (US); Orkan Telhan, Cropseyville, NY (US); Jacob Michael Winiski, Troy, NY (US)

(21) Appl. No.: 18/964,134

(22) Filed: Nov. 29, 2024

Related U.S. Application Data
(60) Provisional application No. 63/604,763, filed on Nov. 30, 2023.
(60) Provisional application No. 63/550,271, filed on Feb. 6, 2024.

(51) Int. Cl. C12M 1/36; C12M 1/00; C12M 1/34; G06N 20/00

(52) U.S. Cl. CPC C12M 41/48; C12M 41/06; C12M 41/12; C12M 41/32; C12M 41/34; C12M 41/36; C12M 41/40; G06N 20/00

(57) ABSTRACT

Systems, methods, and machine learning tools are described for controlled growth of mycelium such as aerial mycelium. Methods can include providing one or more growth environments including one or more inocula and a substrate, providing a monitoring system that transmits data to one or more processors and includes a machine learning tool that monitors environmental conditions and/or mycelium growth, and applying the machine learning tool to determine one or more changes to environmental conditions based on the data. The data can include real-time monitoring information on the environmental conditions or mycelium growth within the one or more growth environments. Further machine learning models are provided for selection and control of additional mycelium growth conditions.

### INCORPORATION BY REFERENCE TO PRIORITY APPLICATIONS

Any and all applications for which a foreign or domestic priority claim is identified in the Application Data Sheet as filed with the present application are hereby incorporated by reference under 37 CFR 1.57. In particular, this application claims filing benefit of U.S. Provisional Patent Application No. 63/604,763 having a filing date of Nov. 30, 2023 and U.S. Provisional Patent Application No. 63/550,271 having a filing date of Feb. 6, 2024, which are hereby incorporated by reference in their entirety.

### BACKGROUND

### Field

This application relates generally to improved methods and systems of growing aerial mycelium, and in particular, for automating conditions within a mycelial growth environment.

### Background

The mycelium is the vegetative part of filamentous fungi, consisting of a network of fine filaments called hyphae. The principal role of a mycelium is to facilitate the growth and nutrient absorption of a fungus. The technological application of mycelia has recently gained significant interest due to their use as or in, e.g., biodegradable material, biofabrication, and environmental remediation.

One promising area of mycelium research has been the production of aerial mycelium, a form of mycelium having unique properties and potential applications, particularly in the food and textile industries. Aerial mycelia refer to hyphae that emerges from a colonized substrate into an airmass directly adjacent to a mycelium. For a solid-state substrate (e.g., source of nutrition, growth substrate or growth matrix), aerial hyphae extend out and away from the substrate or matrix (and in some instances up to several or many inches away from a substrate or matrix, with said substrate or matrix typically being a solid-state, particulate material), as opposed to in and around the substrate or matrix. Such aerial hyphae can be separated as a mass from that solid state growth substrate or matrix as a discrete material, typically without significant additional steps needed to remove stray or embedded substrate or matrix particulate material.

The industrial production of aerial mycelia has required the creation of unique interior growth spaces with equally unique environmental conditions and equipment, in order to promote consistent and successful growth of aerial mycelia on a commercial scale. While various methods and techniques have been developed to control the growth of aerial mycelia, there continues to be a need for more efficient and optimized mycelium growth methods and systems, and aerial mycelium growth methods and systems in particular. For instance, various patent references describe systems and methods for creating mycelium-based materials and structures. These include references directed to both solid state and liquid state fermentation processes. However, these references focus on static controlling of environmental parameters to achieve a desired mycelium crop, focusing on such parameters as temperature, humidity, and substrate composition. For instance, a single condition thermostatic control is used to maintain growth environment temperature within a desired range and period of time. Such limited control precludes dynamic modification in response to evolving mycelial requirements and/or other growth environment parameters, during the growth process, and in response to unexpected environmental or biological fluctuations. There is a need for methods that dynamically control a larger number of mycelium growth parameters, given the rapidly expanding understanding of mycelial growth requirements, the inconsistency of current mycelial growth equipment, and the uncertainties unique to biological manufacturing systems. There is also a need for growth methods and systems that are robust and adaptable to new technologies, e.g., artificial intelligence, to produce materials having consistently desired properties more reliably. Last, there is a need for mycelial growth methods and systems which can dynamically adjust to unexpected environmental fluctuations vis-à-vis biological developments in the fungus being grown, which can be best achieved through real-time observation of mycelium morphology.

While monitoring sensors (e.g., optical sensors/imaging sensors) and condition-reactive devices (e.g., “on” and “shut-off” responsive to static conditions) have been developed and successfully implemented in plant agriculture (e.g., United States Patent Publications 2022/0338420 and 2022/0338421), there is still a need for precise and dynamic growth systems suited for the unique challenges presented by industrial production of fungal mycelia. This need is especially apparent among the currently available computer-driven agricultural automation tools, for which technologies related to the specific needs of fungal mycelia are severely lacking.

### SUMMARY

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, a method for controlling the environmental conditions in one or more growth environments for optimizing mycelium growth is contemplated. The method can include providing one or more growth environments, which can further include one or more fungal inocula and a substrate; providing a monitoring system that transmits data to a processor, which can further include a machine learning tool executing on the one or more processors that monitor at least one environmental condition and/or the mycelium growth in the one or more growth environments; and applying the machine learning tools to determine changes to the environmental conditions based on the data, which can further include real-time monitoring information on the environmental conditions or the mycelium growth within the one or more growth environments.

In various aspects, the one or more growth environments can include at least one control device.

In various aspects, the mycelium can be an aerial mycelium.

In various aspects, the method can further include, under control of the one or more processors, activating at least one of the one or more environmental control devices to implement the one or more changes to the environmental conditions.

In various aspects, the method can include the one or more environmental control devices that can further include at least one of a temperature control device, a mist control device, a relative humidity control device, an atmospheric pressure control device, and an atmospheric gas control device, an airflow control device; the monitoring system can analyze the mycelium growth by image or video sensing of at least one or more of temperature, mist level, mist liquid composition, mist direction, relative humidity, atmospheric pressure, or atmospheric gases, airflow velocity, airflow volume, airflow direction, electromagnetic radiation, visual light, nutritional supplements in the one or more growth environments.

In various aspects, the method can include the machine learning tool that can have been previously trained to recognize optimum mycelium growth conditions. The growth conditions can be selected from one or more datasets that can include at least one of: previous predefined successful growth runs of a same fungal species and/or strain; a selection of preferred accumulated growth conditions for the fungal species and/or strain; and a selection of growth conditions specific to the fungal species and/or strain that can be based on at least one of: the fungal species and/or strain lifecycle, predefined mycelium growth image or video sensing data for a time period in a mycelium growth cycle, predefined mycelium growth image or video sensing data for a select successful growth run of the same fungal species and/or strain, and/or predefined mycelium growth image or video sensing data for a desired mycelium product.

In various aspects, the method can include a monitoring system that can include an image system; the monitoring system can periodically view the growing mycelium for either capturing and later transmitting, or capturing and transmitting in real time, continuous data of the growing mycelium to the processor; and the processor can communicate with the machine learning tool.

In various aspects, the method can include the image system that can further include an imaging or video sensor system in the one or more growth environments for capturing and transmitting either periodic still photographic images or a continuous video feed of the growing mycelium to the processor, which can include the machine learning tool.

In various aspects, the method can include the imaging or video sensor system in the one or more growth environments and can include continuous video feed data; the continuous video feed data can be preprocessed to remove data noise and enhance data quality.

In various aspects, the method can include the machine learning tool being applied to the preprocessed data to perform stable diffusion smoothing while preserving features including at least one of edges or corners.

In various aspects, the method can include the machine learning tool that can further include a Perona-Malik trained model.

In various aspects, the method can include the machine learning tool that can utilize a supervised learning approach to train the model. The model can be trained using the preprocessed data and can include teaching the model to recognize different patterns and structures associated with specific growth stages, growth conditions, deviant mycelium morphologies, or desirable physical attributes previously associated with predefined data patterns.

In various aspects, the method can include the trained model that can be validated by testing the trained model on pre-validated data to assess the accuracy of the trained model in detecting different growth stages, growth conditions, deviant mycelium morphologies or desirable physical attributes previously associated with predefined data patterns.

In various aspects, the method can include the trained model that can continuously analyze the video feed data from the image system and subsequently can analyze and adjust the environmental conditions to promote optimal mycelium growth.

In various aspects, the method can include the trained model that can be monitored and refined based on ongoing analysis and observed growth outcomes to achieve desired growth results.

In various aspects, the method can include the trained model that can be trained to consider downstream production factors such as yield weight, surface topography, flavor-indicating features, presence of color variations, and/or tensile strength.

In various aspects, the method can include the monitoring system that can further include multiple models in evaluating grown mycelium materials.

In various aspects, the method can further include providing supplemental machine learning tool inputs for consideration by the machine learning tool. The inputs can be based on collected growth run data, substrate data or equipment data.

In various aspects, the method can include the one or more growth environments that can further include one or more sub-environments with aerial mycelium at different growth stages. The one or more sub-environments can include the machine learning tool that can be trained to observe how different environmental conditions impact aerial mycelium growth progression by monitoring the environmental conditions at various lifecycle stages of the aerial mycelium.

In various aspects, the method can include the one or more growth environments that can further include two or more fungal species and/or strains, and the machine learning tool can be trained to learn how different growth conditions or different substrates optimize growth of each of the two or more fungal species and/or strains, how different growth conditions or different substrates optimize growth of the two or more fungal species and/or strains when grown together, and/or how different growth conditions or different substrates in different sub-environments optimize growth of the two or more fungal species and/or strains.

In various aspects, the method can include the machine learning tool that can be integrated with intranet or internet query communication tools such that clarification information for continuous improvement of observed data from the image system is accessible to the machine learning tool.

In various aspects, the method can include the machine learning tool, which can be taught to implement learnings from prior stored run successes and failures for future runs.

In another aspect, a method for controlling environmental conditions in a growth environment for aerial mycelium growth is contemplated using an image or video sensing system. The method can include: providing an image or video sensing system in the growth environment for capturing a continuous video feed data of growing and mature aerial mycelium; providing at least one fungal inoculum; providing a substrate for growing the at least one fungal inoculum into aerial mycelium within the growth environment; furnishing the growth environment with equipment necessary to optimize aerial mycelium growth; applying a machine learning tool to the continuous video feed data to perform stable diffusion smoothing while preserving features of the data, such as edges and corners; utilizing a supervised learning approach to train the machine learning tool, teaching the machine learning tool to recognize different patterns and structures associated with specific growth stages, conditions, or deviant morphologies of the aerial mycelium; validating said teaching of the machine learning tool by testing the machine learning tool with validation data to assess accuracy in detecting growth stages, conditions, or the deviant morphologies of aerial mycelium; using the trained machine learning tool to continuously analyze the continuous video feed from the image or video sensing, and to adjust the environmental conditions that can be selected from a group consisting of: temperature, relative humidity, misting levels, misting liquid composition, misting direction, air flow, gaseous components, in order to promote optimized mycelium growth.

In various aspects, the method can include the machine learning tool, wherein the machine learning tool can implement a Perona-Malik model to perform stable diffusion smoothing while preserving important features of the data, such as edges and corners.

In various aspects, the method can include preprocessing the continuous video feed data to remove noise and to enhance video quality.

In various aspects, the method can include selecting mature aerial mycelium in accordance with predefined mycelium physical attributes, such as one selected from the group comprising yield weight, surface topography, flavor-indicating features, presence of color variations, and tensile strength.

In another aspect, a method for controlling environmental conditions for aerial mycelium growth in one or more growth environments contemplates using a dynamic computer image system. The method can include: providing one or more growth environments with equipment to grow aerial mycelium; providing one or more fungal inocula; providing substrate adjacent to the one or more fungal inocula within the growth environment and allowing the one or more fungal inocula to grow into the aerial mycelium; providing an image or video sensing system in the growth environment to capture continuous video feed data of the growing mycelium; utilizing a Perona-Malik model to perform stable diffusion smoothing of the images captured by the image or video sensing system; modulating at least one environmental condition selected from the group comprising temperature, humidity, airflow, relative humidity, misting levels, misting rate, misting liquid composition, misting direction, gaseous content, and atmospheric pressure, over time, while continuously recording image or video sensing data of the growing aerial mycelium; using a machine learning model to continuously control the at least one environmental condition to optimize the aerial mycelium growth, the model selected from the group comprising deep neural networks, or decision trees; incorporating a feedback loop in the machine learning model to enable real-time adjustment of environmental conditions during at least one of a 7 day to 16 day, 7 day to 14 day, or 10 day to 14 day growth run to optimize the aerial mycelium growth, and; continuously monitoring the aerial mycelium growth and adjusting said environmental conditions based on real-time analysis of the continuous video feed data to control growth environment inputs and outputs.

In various aspects, the method can include the growth environment inputs, which can further include at least one of nutrients, temperature, relative humidity, misting levels, misting liquid composition, misting direction, air flow, or gaseous components.

In various aspects, the method can include the growth environment that can further include outputs that can include at least one of mature aerial mycelium, immature aerial mycelium with desired morphologies and/or properties, or deviant aerial mycelium morphologies.

In various aspects, the method can include refining the trained model based on real-time analysis of the outputs to optimize said environmental conditions for optimized mycelium growth.

In another aspect, a method is contemplated for substrate selection for growing aerial mycelium using a machine learning tool. The method can include: providing a machine learning tool; training the machine learning tool by providing the machine learning tool with previously collected data on optimizing aerial mycelium growth; and selecting the substrate that meets the nutritional requirements for growing specific fungal species and/or strains based on analyses by the machine learning tool.

In various aspects, the method can include the previously collected data on optimizing aerial mycelium growth, an can further include: previously identified nutritional requirements for growing specific fungal species and/or strains; previously identified plant sources for providing the previously identified nutritional requirements for growing specific fungal species and/or strains, and/or; identified local plant sources by geographical region providing the previously identified nutritional requirements for growing specific fungal species and/or strains.

In various aspects, the method can include the previously collected data on optimizing aerial mycelium growth, which can further include determining by experimentation the nutritional requirements of a desired one or more fungal species and/or strain.

In another aspect, a method is contemplated for substrate selection for growing aerial mycelium using a machine learning tool. The method can include: providing a machine learning tool; training the machine learning tool by providing the machine learning tool with previously collected data on optimizing aerial mycelium growth; and determining, based on one or more predetermined target morphologies for the aerial mycelium, one or more desired nutritional profiles for the substrate.

In various aspects, the method can include the previously collected data on optimizing aerial mycelium growth, which can further include experimentally collected data associating determined aerial mycelium morphologies and corresponding substrate nutritional profiles.

In another aspect, a system for controlling aerial mycelium growth is contemplated. The system can include: at least one growth environment configured to continuously monitor environmental conditions and/or mycelium growth; and one or more processors configured by computer-executable instructions stored in a non-transitory medium to execute a machine learning tool based on the environmental conditions and/or mycelium growth within the at least one growth environment, the one or more processors can be further configured to at least: capture continuous feed image or video sensing data of the growth environment, the sensing data representing current and real-time environmental conditions; preprocessing the sensing data to remove data noise and enhance data quality by performing stable diffusion smoothing of the sensing data while preserving important features, including at least one of edges or corners; analyze said sensing data using the machine learning tool to determine one or more adjustments to the environmental conditions; and adjust the environmental conditions to implement the determined one or more adjustments by controlling one or more environmental control devices within the at least one growth environment.

In various aspects, the system can include the one or more adjustments that can further include an adjustment to at least one of temperature, relative humidity, misting levels, misting liquid composition, misting direction, air flow, and gaseous components, in order to promote a desired mycelium growth.

In various aspects, the system can include the machine learning tool that can further include maintaining or modifying the environmental conditions to a desired level for optimizing aerial mycelium growth by using a supervised machine learning approach to train the machine learning tool to recognize different patterns and structures associated with specific growth stages, conditions, or morphologies of said aerial mycelium.

In another aspect, a method for controlling environmental conditions of one or more growth environments for optimizing mycelium growth is contemplated. The method can include: providing the growth environment that can include one or more fungal inocula and a substrate; providing within the growth environments one or more proxy bioreactor models, wherein said one or more proxy bioreactor models can include an aerial mycelium growth vessel that provides location-specific information on environmental conditions and mycelium growth within the growth environment; providing a monitoring system that transmits data to one or more processors, wherein the monitoring system can include a machine learning tool executing on the one or more processors that can monitor at least one of the one or more proxy reactor models; and applying the machine learning tool to determine one or more changes to the environmental conditions based on the data, wherein the data comprises real-time monitoring information on the environmental conditions or the mycelium growth within the growth environment.

In various aspects, the method can include the one or more growth environments that can further include one or more environmental control devices.

In various aspects, the method can further include, under control of the one or more processors, activating at least one of the one or more environmental control devices to implement the one or more changes to the environmental conditions.

In another aspect, another method for controlling environmental conditions of one or more growth environments for optimizing mycelium growth is contemplated. The method can include: providing the growth environment that can further include one or more fungal inocula and a substrate; providing within the growth environments one or more voxels, wherein said one or more voxels can include a three-dimensional virtual grid overlay of the growth environment, and wherein the one or more voxels can further include at least one virtual cell that can include a volume of at least one of air, airflow, mist, mycelium, or substrate; providing a monitoring system that can transmit data to one or more processors, wherein the monitoring system includes a machine learning tool executing on the one or more processors that can monitor at least one of the voxels; and applying the machine learning tool to determine one or more changes to the at least one cell based on the data, wherein the data comprises real-time monitoring information on the at least one cell.

In another aspect, a method for controlling environmental conditions of one or more growth environments for optimizing mycelium growth, while allowing selection of mature mycelium for one or more predetermined uses is contemplated. The method can include: providing one or more growth environments that can further include one or more fungal inocula and a substrate; providing a monitoring system that can transmit data to one or more processors, wherein the monitoring system can include a machine learning tool executing on the one or more processors that can monitor at least one of environmental conditions and mycelium growth in the one or more growth environments; and applying the machine learning tool to determine one or more changes to the environmental conditions based on the data, wherein the data can include real-time monitoring information on the environmental conditions or the mycelium growth within the one or more growth environments.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

The features and advantages of the methods, systems, and compositions described herein will become more fully apparent from the following description and appended claims, taken in conjunction with the accompanying drawings. These drawings depict only several embodiments in accordance with the disclosure and are not to be considered limiting of their scope. In the drawings, similar reference numbers or symbols typically identify similar components, unless context dictates otherwise. In some instances, the drawings may not be drawn to scale.

FIG. 1A illustrates an embodiment of a growth matrix in a first exemplary growth environment, suitable to support extra-particle aerial mycelial growth.

FIG. 1B illustrates an embodiment of extra-particle aerial mycelial growth extending from the growth matrix of FIG. 1A in said growth environment.

FIG. 2A illustrates an additional exemplary embodiment of a growth environment (growth chamber) with multiple shelves of trays growing aerial mycelium.

FIG. 2B illustrates a third exemplary embodiment of a portion of a growth environment (growth chamber) with multiple shelves for trays or beds for growing aerial mycelium.

FIG. 3A illustrates a block diagram of aerial mycelium growth system architecture for an aerial mycelium growth environment according to one embodiment.

FIG. 3B illustrates generalized exemplary\ AI model embodiments for use with the aerial mycelium growth system architecture of FIG. 3A.

FIG. 4 illustrates an exemplary method for running an aerial mycelium growth chamber in accordance with one embodiment of the invention.

FIG. 5 illustrates still another exemplary method for running an aerial mycelium growth chamber in accordance with one embodiment of the invention.

FIGS. 6A and 6B show overhead photographic images of aerial mycelium material grown in a tray format, with a generally non-bumpy, homogeneous surface.

FIGS. 7A and 7B shows overhead photographic images of aerial mycelium material grown in a tray format, with a generally bulbous surface.

FIG. 8 shows an overhead photographic image of a recently inoculated and incubated growth matrix in a bed of a growth environment with initial growth of aerial mycelium partially evident, and areas of exposed growth matrix likewise partially evident.

FIG. 9 shows an overhead photographic image of an almost fully incubated, growth matrix in a bed, with growth matrix no longer visible and with contiguous mature aerial mycelium growing or grown across the entire growth matrix and having a generally flat, homogenous surface.

FIG. 10 illustrates a representation image of a proxy reactor in accordance with one embodiment of the invention, which proxy reactor may itself utilize grown aerial mycelium as a proxy sensor in a relatively large growth environment as part of an AI model.

FIG. 11 illustrates a representation of proxy reactor digital twins, showing conditions and interpolated predictions, a digital twin proxy reactor and a heat map.

FIG. 12 illustrates a representation of a proxy reactor network and associated localized data reporting.

FIG. 13 illustrates an exemplary embodiment of the invention showing a diagrammatic view of a goal-driven AI model.

FIG. 14 illustrates an exemplary embodiment of the invention, showing the mechanics of a research AI model and design of experiments AI model, for use in optimizing a growth trajectory of aerial mycelium.

FIG. 15 illustrates a representation of a voxel AI model (spatial configuration model considering the air, fiber, and substrate spaces in a growth environment) for use with the invention.

FIG. 16 illustrates a flow diagram of a substrate defining AI model for use with the invention.

### DETAILED DESCRIPTION

U.S. Pat. No. 11,277,979 to Greetham et al., International PCT Patent Application No. WO2019/099474A1 to Kaplan-Bie et al., PCT Patent Application No. WO2023172696 to Snyder et al., PCT Patent Application No. WO2022235688 to Winiski et al., and PCT Patent Application No. WO2022235694 to Carlton et al., the entirety of each of which are incorporated herein by reference thereto except where inconsistent with the disclosure herein, describe methods of growing a mycological biopolymer material, aerial mycelium and products resulting therefrom, including environmental conditions in which such aerial mycelium materials may be grown. The methods of the current disclosure utilize environmental conditions as described in the preceding references, to provide more consistently repeatable growth systems, that are adaptable to changing environmental conditions, adaptable to accommodate the needs of varying fungal organisms, adaptable to different growth equipment paradigms, and also adaptable to target differing attributes desired in various mycelium products. Still further the disclosed systems offer the potential for energy and resource efficiency as a result of closely monitored conditions and thoughtful action steps taken in relation thereto (after considering multiple implications and variables) while still providing high quality and quantity mycological (e.g., mycelium-based) material.

Described herein are embodiments of systems, modified methods, and apparatuses to consistently grow aerial mycelium material (mycelia grown through a solid-state fermentation process), but in certain instances, also other mycelial materials grown through a liquid (surface) fermentation process. The aerial and other mycelium material that is grown via such systems can be used in the food industry (for example, as an animal-based, meat-substitute product, and one that may present to the consumer a product that offers the appearance and texture of traditional, animal-based meat material (e.g., beef, pork, poultry and seafood), and in other industries, such as textiles, packaging, and others.

It is a further object of the present invention to provide mycelial growth methods that offer flexibility such that the methods are capable of toggling back and forth between various mycelial growth step options (among several available, such as in sequence of steps, or overall steps utilized), or alternatively that are adaptable to the differing requirements of various mycelial organism strains as to accommodate either varying product configurations (with each design demonstrating differing desired end-product attributes), or to accommodate various manufacturing/growth facility spaces or growth equipment availability.

It is still a further object of the present invention to provide modified mycelial growth methods and systems (such as for example, “feedback loop” systems with real time adaptive control and feedback from processors, to various equipment systems within a growth environment) which utilize machine learning tools to adapt to changing environmental conditions of growth environments, unforeseen organismal responses, or changing product configurations, or alternative manufacturing geographic locations (based on different substrate availabilities), or changing substrates, or direct observations of partially-grown or fully-grown aerial mycelium material (such as to modify current or future growth cycles, or to select and direct usage of grown aerial mycelium material to appropriate uses in accordance with predefined product attributes.

It is a further object of the present invention to provide methods and systems for growing mycelium that are consistent, repeatable, adaptable to various technology platforms, and energy efficient, while providing for predictably high quality and quantity mycelium-based materials that are useful, practical, and which may be produced at any scale, from small-scale benchtop biological reactors to large-scale manufacturing facilities. The methods and systems can include growing mycelia in one or more growth environments; maintaining one or more conditions of the growth environment, with at least one condition selected from a predetermined growth atmosphere of temperature, relative humidity, atmospheric gas content sufficient to produce a mycelium, airflow rates and/or directions, mist levels, composition and/or direction, light levels, moisture content and nutrition source availability; and operating or “running” the growth environment for a period of time sufficient to produce the desired mycelium (e.g., a desired mature aerial mycelium). The mycelium can comprise, consist essentially of, or consist of fungal mycelium. The mycelium can comprise, consist essentially of, or consist of aerial mycelium. The mycelium can comprise, consist essentially of, or consist of mature mycelium.

It is another object of the present invention to provide methods and systems of growing mycelium that comprise at least a solid-state fermentation process. Aspects of such methods and systems for use with solid-state fermentation processes may also be suitable, in accordance with several embodiments of the present description, for use in manufacturing processes based on liquid fermentation and submerged fermentation processes.

It is a further object of the invention to provide for growth methods and systems for mycelia grown through other fermentation processes, for example, liquid surface fermentation processes, and submerged fermentation processes.

It is another object of the invention to provide efficient and precise mycelium growth control systems and associated methods, that can monitor mycelial growth, and through a machine learning tool, adapt environmental conditions to the growth progress of the mycelium in real time, based on at least one of predetermined set of target morphologies or appearances, and/or predetermined condition ranges, and/or predetermined favored success conditions based on desired fungal strains.

It is another object of the invention to provide a system and method for controlling and optimizing aerial mycelium growth using one or more image monitoring systems and one or more machine learning tools.

It is another object of the invention to provide autonomous growth systems for aerial mycelium that leverage machine learning tools to dynamically change growth conditions based on a wide array of prior or continuing growth experiential data, and/or selective human training.

It is still a further object of the invention to provide a “feedback loop” learning manufacturing system in the mycelium field, for preparing substrate for particular strains of fungus, and growing mycelium (or processing it) so as to create an efficient and adjustable manufacturing platform.

It is yet a further object of the invention to provide a feedback loop control system which utilizes diffusion type modeling systems, in that such models would initially be trained, and then would be utilized to control mycelial growth in growth chambers. Such systems would utilize continuous feedback control over already understood and deemed critical growth parameters (which would evolve over time with increased data access) of the growing mycelium, ideally based on a visual target previously set, and through the continuous use of image systems such as image sensors or systems.

It is yet another aspect of the invention to utilize automated analytical methods and visual observations for determining the presence of desired or undesired physical attributes in aerial mycelium, for the ultimate purposes of either alteration of environmental growth conditions in a current mycelial growth run or future run, alternatively, for the purposes of mycelial material selection as being particularly suitable for set uses (from a range of physical attributes that may be visually observed, after visual features have previously been correlated to defined physical attributes such as tensile strength, taste and what not), or alternatively for early disposal of growing mycelium material or rerouting of either lower performing substrate or lower growth levels which demonstrates such deviant morphology making it unsuitable for continued growth and use as an aerial mycelium product (thereby saving energy and resources for later growth runs).

It is still a further object of the invention to provide a system and method for controlling and optimizing aerial mycelium growth using an image system and a machine learning tool which relies on multiple data sources, including at least one selected from: (1) past growth cycle data for the fungal organism in question, (2) identified target values for growth of the fungal organism (such as yield, surface topography, color), (3) identified growth conditions for the fungal organism in question, based on various fungal growth timelines and/or events, especially those conditions associated with predefined successful growth cycles, and (4) variations in growth environments or cycles associated with different fungal organisms.

It is still another object of the invention to provide a system and method for optimizing substrate selection for growth of aerial mycelium using a machine learning tool which relies on multiple data sources. Such data sources can be based on experimental data of previously identified nutritional requirements for growing specific species and/or strains of fungi or any other organism suitable to be grown by the methods and systems disclosed herein. Such data sources can also be identified in-situ based on locally available nutritional sources that can be used by specific species and/or strains of fungi or any other organism suitable to be grown by the methods and systems disclosed herein. The nutritional requirements of different fungal species and/or strains varies significantly; however, plant material can be an important source of nutrition for growing mycelium. Other nutritional requirements of species and/or strains of fungi or any other organism suitable to be grown by the methods and systems disclosed herein can also or additionally derive from animal-based debris or refuse (e.g., chicken manure) or any other organic source of nutrition. The nutritional requirements of species and/or strains of fungi or any other organism suitable to be grown by the methods and systems disclosed herein can also or additionally derive from inorganic sources (e.g., gypsum). Nutritional requirement ranges for growing species and/or strains of fungi or any other organism suitable to be grown by the methods and systems disclosed herein can be determined from physical experimentation. Data derived from physical experimentation can include information on environmental variance, undefined substrate composition variance, and defined nutritional composition variance where the defined nutritional profile is a function of the undefined substrate composition.

It is a further object of the invention to provide methods and systems for optimizing substrate selection for growth of aerial mycelium using a machine learning tool that relies on multiple data sources of available plant resources. The nutritional requirements for growing mycelium can be met by any organic or inorganic nutritional source whose nutritional profile meets the nutritional needs of the desired organism to be grown. Alternatively, the machine learning tool can be used to identify any organic or inorganic nutritional source based on previous experimental data or based on third-party or in-house databases of geographically local organic and inorganic nutritional sources that meet the nutritional needs of a desired organism. For example, the machine learning tool can be trained to identify or interface with digitally available or internet-based flora guides to identify locally available plants that would satisfy the nutritional needs of the species and/or strains of fungi or any other organism suitable to be grown by the methods and systems disclosed herein.

It is still a further object of the invention to provide a system and method for the present invention that addresses this need by providing a feedback loop vision or imaging control system that utilizes an image sensor-setup and one or more machine learning algorithms to monitor and control aerial mycelium growth. The system captures images of the growing mycelium and compares them to a predefined target morphology or appearance. Based on this comparison, the system adjusts environmental parameters in real-time to achieve the desired growth pattern or morphology.

It is still an additional object of this invention to use machine learning models (AI models) selected from the group consisting of goal-driven models, proxy bioreactor models, check and adjust models, MDA models, spatial configuration models (aka voxel models), layered research models, substrate selection models, and combinations thereof.

### Definitions

“Mycelium” as used herein refers to a connective network of fungal hyphae, with mycelia being the plural form of mycelium.

“Hyphae” or “hypha” as used herein refers to branched filament vegetative cellular structures that are interwoven to form mycelium.

“Fruiting body” as used herein refers to a fungal stipe, pileus, gill, pore structure, or a combination thereof, and may be referred to herein as “mushroom.”

“Substrate” as used herein refers to a material or surface thereof, from or on which an organism lives, grows, and/or obtains its nourishment. In some embodiments, a substrate provides sufficient nutrition to the organism under target growth conditions such that the organism can live and grow without providing the organism a further source of nutrients. A variety of substrates are suitable to support the growth of an aerial mycelium of the present disclosure. Suitable substrates are disclosed, for example, in United States Publication 20200239830A1 to O'Brien et al., the entire contents of which are hereby incorporated by reference in their entirety to the extent not inconsistent with the content of this disclosure. In some embodiments, the substrate is a natural substrate. Non-limiting examples of a natural substrate include a lignocellulosic substrate, a cellulosic substrate, or a lignin-free substrate. A natural substrate can be an agricultural waste product or one that is purposefully harvested for the intended purpose of food production, including mycelial-based food production. Further non-limiting examples of substrates suitable for supporting the growth of mycelia of the present disclosure include soy-based materials, oak-based materials, maple-based materials, corn-based materials, seed-based materials and the like, or combinations thereof. The materials can have a variety of particle sizes, as disclosed in US20200239830A1, and occur in a variety of forms, including shavings, pellets, chips, flakes, or flour, or can be in monolithic form. Non-limiting examples of suitable substrates for the production of mycelia of the present disclosure include corn stover, maple flour, maple flake, maple chips, soy flour, chickpea flour, millet seed flour, oak pellets, soybean hull pellets and combinations thereof. Additional useful substrates for the growth of mycelia are disclosed herein. A substrate can also be a depleted substrate, which is at least partially depleted of nutrients or other materials after extra-particle aerial mycelial growth has been grown and divided from the growth matrix to form a separated aerial mycelium. A substrate or a depleted substrate can be a substrate which has been further processed (e.g., chemically or mechanically) to improve its viability to support new mycelial growth (e.g., extra-particle aerial mycelial growth).

“Growth media” or “growth medium” as used herein refers to a matrix containing a substrate and an optional further source of nutrition that is the same or different than the substrate, wherein the substrate, the nutrition source, or both are intended for fungal consumption to support mycelial growth.

“Growth matrix” as used herein refers to a matrix containing a growth medium and a fungus. In some embodiments, the fungus is provided as a fungal inoculum; thus, in such embodiments, the growth matrix comprises a fungal-inoculated growth medium. In other embodiments, the growth matrix comprises a colonized substrate.

“Inoculated substrate” as used herein refers to a substrate that has been inoculated with fungal inoculum. For example, an inoculated substrate can be formed by combining an uninoculated substrate with a fungal inoculum. Alternatively, an inoculum can be a solid or liquid composition of any living organism or part thereof, including, but not limited to, bacteria, archaea, viruses, protozoa, algae, animal cells or tissue, plant cells or tissue, or any other living material.” An inoculated substrate can be formed by combining an uninoculated substrate with a previously inoculated substrate. An inoculated substrate can be formed by combining an inoculated substrate with a colonized substrate.

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth media substrate. The colonized substrate may further contain residual nutrition that has not been consumed by the colonizing fungus. As is understood by persons of ordinary skill in the art, a colonized substrate has undergone primary myceliation, sometimes referred to by skilled artisans as having undergone a “mycelium run.” Thus, in some particular aspects, a colonized substrate consists essentially of a substrate and a colonizing fungus in a primary myceliation phase. For many fungal species, asexual sporulation occurs as part of normal vegetative growth, and as such could occur during the colonization process. Accordingly, in some embodiments, a colonized substrate of the present disclosure may also contain asexual spores (conidia). In some aspects, a colonized substrate of the present disclosure can exclude growth progression into sexual reproduction and/or vegetative foraging. Sexual reproduction includes fruiting body formation (e.g., primordiation and differentiation) and sexual sporulation (meiotic sporulation). Vegetative foraging includes any mycelial growth away from the colonizing substrate (such as aerial growth). Thus, in some further aspects, a colonized substrate can exclude mycelium that is in a vertical expansion phase of growth. A colonized substrate can enter a mycelial vertical expansion phase during incubation in a growth environment of the present disclosure. For example, a colonized substrate can enter a mycelial vertical expansion phase upon introducing aqueous mist into the growth environment and/or depositing aqueous mist onto colonized substrate and/or any ensuing extra-particle growth. In some embodiments, the use of aqueous mist can be adjusted, for example, to desired levels, direction, composition, and timing, to affect the topology, morphology, density, and/or volume of the growth. In some further embodiments, mist can be comprised of two or more liquid compositions. For example, introduction of liquid mist can be sourced from reservoirs of liquid water, liquid nutrients, liquid dye, liquid flavoring, liquid texturizing solutions, liquid tenderizing solutions, liquid mineral solutions, or any other liquid solution that can affect the topology, geometry and/or morphology of aerial mycelium.

Any suitable substrate can be used alone, or optionally combined with a nutrient source, as media to support mycelial growth. The growth media can be hydrated to a final target moisture content prior to inoculation with a fungal inoculum. In a non-limiting example, the substrate or growth media can be hydrated to a final moisture content of at least about 50% (w/w), at most about 95% w/w, within a range of about 50% to about 95%. Growth media hydration can be achieved via the addition of any suitable source of moisture. In a non-limiting example, the moisture source can be airborne or non-airborne liquid phase water (or other liquids), an aqueous solution containing one or more additives (including but not limited to a nutrient source), and/or gas phase water (or other compound). In some embodiments, at least a portion of the moisture is derived from steam utilized during bioburden reduction of the growth media. In some embodiments, inoculation of the growth media with the fungal inoculum can include a further hydration step to achieve a target moisture content, which can be the same or different than the moisture content of the growth media. For example, if growth media loses moisture during fungal inoculation, the fungal inoculated growth media can be hydrated to compensate for the lost moisture.

Methods for the production of aerial mycelium disclosed herein can include an inoculation stage, wherein an inoculum is used to transport an organism into a substrate. The inoculum, which carries a desired fungal strain, is produced in sufficient quantities to inoculate a target quantity of substrate. The inoculation can provide a plurality of myceliation sites (nucleation points) distributed throughout the substrate. Inoculum can take the form of a liquid, a slurry, or a solid, or any other known vehicle for transporting an organism from one growth-supporting environment to another. Generally, the inoculum comprises water, carbohydrates, sugars, vitamins, other nutrients, and at least one fungus. The inoculum may contain enzymatically available carbon and nitrogen sources (e.g., lignocellulosic biomass, chitinous biomass, carbohydrates) augmented with additional micronutrients (e.g., vitamins, minerals). The inoculum can contain inert materials (e.g., perlite). In a non-limiting example, the fungal inoculum can be a seed-supported fungal inoculum, a feed-grain-supported fungal inoculum, a seed-sawdust mixture fungal inoculum, or another commercially available fungal inoculum, including specialty proprietary spawn types provided by inoculum retailers. In some aspects, a fungal inoculum can be characterized by its density. In some embodiments, a fungal inoculum has a density of about 0.1 gram per cubic inch to about 10 grams per cubic inch, or from about 1 gram per cubic inch to about 7 grams per cubic inch. A skilled person can modify variables including the substrate or growth media component identities, substrate or growth media nutrition profile, substrate or growth media moisture content, substrate or growth media bioburden, inoculation rate, and inoculum constituent concentrations to arrive at a suitable medium to support aerial mycelial growth.

“Growth environment” as used herein refers to an environment that supports the growth of mycelia, as would be readily understood by a person of ordinary skill in the art in the mycelial cultivation industry, which contains a growth atmosphere having a gaseous environment of carbon dioxide (CO₂), oxygen (O₂), and a balance of other atmospheric gases including nitrogen (N₂), and which is further characterized as having a relative humidity. In some aspects of the present disclosure, the growth atmosphere can have a CO₂ content of at least about 0.02% (v/v), at least about 0.6%, at least about 5% (v/v), less than about 10% (v/v), less than about 8% (v/v), less than about 7%, between about 0.02% and 10%, between about 0.02% and 8%, between about 0.6% and about 7%, between about 5% and about 10%, or between about 5% and about 8%. In some other aspects, the growth atmosphere can have an O₂ content of at least about 12% (v/v), or at least about 14% (v/v), and at most about 21% (v/v). In yet other aspects, the growth atmosphere can have an N₂ content of at most about 79% (v/v). Each foregoing CO₂, O₂ or N₂ content is based on a dry gaseous environment, notwithstanding the growth environment atmosphere relative humidity. “A portion of the growth environment” as used herein refers to a percentage of the total volume of the growth environment. For example, a portion of the growth environment can encompass between 0.01% to 100% of the total volume of the growth environment. A portion of the growth environment can refer to any fraction of the one-dimensional, two-dimensional or three-dimensional geometry comprising the growth environment. For example, a portion of the growth environment can refer to the unit length, the unit width, the unit height, the unit body diagonal, the unit face diagonal, the unit perimeter, the unit radius, the unit circumference, the unit surface area, the unit cross section, or the unit volume of the growth environment.

The geometry of the growth environment can be customized to support mycelium growth at several spatial scales. In some embodiments, the volume of the growth environment can fall within a range of between about at least 0.1 ft³ and/or less than or equal to about 500,000 ft³. or can fall within a range between about at least 1.0 ft³ and/or less than or equal to 250,000 ft³. In some yet further embodiments, the volume of the growth environment can be about 0.1 ft³, 0.2 ft³, 0.3 ft³, 0.4 ft³, 0.5 ft³, 0.6 ft³, 0.7 ft³, 0.8 ft³, 0.9 ft³, 1.0 ft³, or any range therebetween. In some yet further embodiments, the volume of the growth environment can be about 250,000 ft³, 300,000 ft³, 400,000 ft³, 500,000 ft³, or any range therebetween.

A growth environment can comprise one or more sub-environments. For example, each sub-environment can be comprised of aerial mycelium at different growth stages. In some embodiments, the one or more sub-environments can include a machine learning tool that can be trained to observe how different environmental conditions impact aerial mycelium growth progression in one or more sub-environments. For example, the machine learning tool can monitor the environmental conditions at various lifecycle stages of the aerial mycelium in one or more sub-environments. In some further embodiments, one or more sub-environments can be monitored by the machine learning tool and dynamically adjusted to optimize the aerial mycelium growth in a sub-environment. For example, the machine learning tool can anticipate the likelihood of one or more aerial mycelium properties given already recorded data of aerial mycelium growth. As a further non-limiting example, different aesthetic and mechanical properties can be anticipated in real-time to forecast particular uses of the mycelium.

“Aerial mycelium” as used herein refers to mycelium obtained from extra-particle aerial mycelial growth, and which is substantially free of growth matrix (and is that part of mycelial growth that extends away from and apart from a substrate or growth matrix).

“Mature mycelium” as used herein refers to mycelium that is still in contact with the growth medium, growth media, or substrate and is suitable for use. In some embodiments, the mature mycelium can be mature aerial mycelium.

“Extra-particle mycelial growth” (EPM) as used herein refers to mycelial growth, which can be either appressed or aerial.

“Extra-particle aerial mycelial growth” as used herein refers to a distinct mycelial growth that occurs away from and outward from the surface of a growth matrix. Extra-particle aerial mycelial growth can exhibit negative gravitropism. In a geometrically unrestricted scenario, extra-particle aerial mycelial growth could be described as being positively gravitropic, or neutrally gravitropic, aerial, and radial in which growth will expand in all directions from its point source. In some embodiments, external forces, such as airflow, can be applied towards (e.g., approximately perpendicular to the growth environment floor) the growth substrate, and in some embodiments, through the growth substrate, for example, to create downward aerial mycelium growth in the direction of gravity. Alternatively, airflow can be applied across the growth substrate in a manner parallel or horizontal to the growth substrate surface.

“Positive gravitropism” as used herein refers to growth that preferentially occurs in the direction of gravity.

“Negative gravitropism” as used herein refers to mycelial growth that preferentially occurs in the direction away from gravity. As disclosed herein, extra-particle aerial mycelial growth can exhibit in one embodiment negative gravitropism. Without being bound by any particular theory, this may be attributable at least in part to the geometric restriction of the growth format, wherein an uncovered tool having a bottom and side walls contains a growth matrix. With such geometric restriction, growth will primarily occur along the unrestricted dimension(s), which in the scenario is primarily vertically (negatively gravitropic).

“Growth run” or “run” as used herein refers to the time period under specific environmental conditions during which a mature mycelium is formed. In some embodiments, a growth run or “run” can be synonymous with or comprise a period of incubating. Aerial mycelia of the present disclosure can be grown in a matter of weeks or days. In some embodiments, a growth run is of a duration between about 10 days and 164 days, alternatively between about 10 days and 14 days. This feature is of practical value in the production of food ingredients or food products, where time and efficiency are at a premium. Accordingly, the presently disclosed method of making an aerial mycelium comprises incubating a growth matrix in a growth environment for an incubation time period of up to about 3 weeks. In some embodiments, the incubation time period can be within a range of about 4 days to about 17 days. In some further embodiments, the incubation time period can be within a range of about 7 days to about 16 days, within a range of about 8 days to about 15 days, within a range of about 9 days to about 15 days, within a range of about 9 days to about 14 days, within a range of about 8 to about 14 days, within a range of about 7 to about 13 days, or within a range of about 7 to about 10 days. In some more particular embodiments, the incubation time period can be about 7 days, about 8 days, about 9 days, about 10 days, about 11 days, about 12 days, about 13 days, about 14 days, about 15 days or about 16 days, or any range therebetween.

Advantageously, incubating a growth matrix comprising a colonized substrate (wherein said colonized substrate comprises a growth medium previously colonized with mycelium of a fungus) in a growth environment of the present disclosure can result in earlier expression of aerial mycelial tissue compared to incubation of a growth matrix comprising substantially the same or a similar growth medium and a fungal inoculum, wherein the fungal inoculum contains a fungus. Accordingly, a method of making an aerial mycelium of the present disclosure can comprise incubating a growth matrix comprising a colonized substrate (wherein said colonized substrate comprises a growth medium previously colonized with mycelium of a fungus) in a growth environment for an incubation time period, and producing extra-particle aerial mycelial growth therefrom, wherein the incubation time period is at least about 1 day, at least about 2 days, at least about 3 days, or at least about 4 days less than the incubation time period for producing extra-particle aerial mycelial growth from a growth matrix comprising a growth medium and a fungal inoculum, wherein the fungal inoculum comprises a fungus.

In some other embodiments, the incubation time period ends no later than when a visible fruiting body forms. In a non-limiting example, the incubation time period can end prior to a karyogamy or meiosis phase of the fungal reproductive cycle. In some other embodiments, the incubation time period ends when a visible fruiting body forms. As disclosed herein, aerial mycelia of the present disclosure can be prepared without the formation of a visible fruiting body, thus, in some embodiments, an incubation time period can end without regard to the formation of a visible fruiting body. Trial incubation runs can be used to inform the period of time in the growth environment during which sufficient extra-particle aerial mycelial growth product occurs (e.g., aerial mycelial growth of a predetermined thickness) without the formation of visible fruiting bodies.

In some embodiments, a method of making an aerial mycelium of the present disclosure can comprise periodically monitoring the growth/morphology of aerial mycelium at various growth stages, and/or controlling certain aspects of a growth environment, including gas content, atmospheric pressure, temperature, relative humidity, mist levels, mist composition, mist direction, lighting, provided nutrients in the form of mist and/or substrate, and nutrient and inert substrate (or growth matrix) content, in response to various monitor readings and aerial mycelium morphology.

“Mycelium-based” as used herein refers to a composition substantially comprising mycelium.

“Real time” or “real-time” as used herein refers to a system in which input data is processed as it is received. In some embodiments, an in-progress mycelium growth cycle or run can be monitored in real time as it is running. In some cases, real-time input data may be processed within a short period of time such as seconds or milliseconds so that it is available virtually immediately as feedback. A mycelium growth cycle refers to an actively used growth environment (such as a growth chamber or bench top reactor) that is currently in the process of growing aerial mycelium, as opposed to a growth environment (e.g., growth chamber or bench top reactor) which is not in use to grow aerial mycelium.

“Machine learning tool” as used herein refers to an algorithmic application of artificial intelligence (AI) that gives systems the ability to learn and improve without ample or significant human input. Human input may be part of the learning for such tools (or models applied through such tools). Such tools may encompass predictive modeling using both historical and real time data, which allows software to become more accurate in predicting outcomes without being explicitly programmed. Such tools often run models in order to optimize decisions at commercial scale.

“Model,” “Artificial Intelligence (AI) Model,” or “machine learning model” as used herein refers to a program that has been trained on a set of data to recognize certain patterns, learn relations between variables, or to make certain decisions without human intervention. In some embodiments, intervention is in some way built into the model or desired by the human operator(s). In some embodiments, AI models apply algorithms to data input to achieve a desired result or output. In some further embodiments, the desired result or output is predetermined by the human operator(s). In an example embodiment, the desired result or output is unknown a priori and the AI model is configured to deliver an optimized result or output. For example, an AI model can be configured to produce aerial mycelium with a desired set of properties for which the optimal environmental conditions are unknown. For example, the desired properties of the mycelium can include tissue density, homogencity, roughness, regularity, uniformity, or any other similar property. The human operator can configure such AI model to determine the unknown optimal environmental conditions in order to achieve the desired aerial mycelium properties. In some further embodiments, image or video sensing data can be optimized using an AI model. For example, the Perona-Malik can be used to process computer vision data and anisotropic diffusion (e.g., Perona-Malik diffusion), to reduce image data noise without removing significant parts of the image content, typically edges, lines or other details that are important for the interpretation of the image.

“Noise” or “data noise” as used herein refers to variations in digital information such as random variation of brightness or color information in images or video data. In some further embodiments, noise can comprise operational failure in any software, hardware or electronics of the present disclosure. For example, noise can be produced by a faulty image or video sensor and/or the circuitry therein. In some further embodiments, noise can be the product of environmental conditions or growth environment malfunction. For example, in some nonlimiting embodiments, monitoring the growth environment using an image or video sensor positioned externally therefrom can result in data noise caused by reduced visibility as caused by, e.g., airborne mist. In some further embodiments, it is desired to provide image model tolerances to discount said environmental noise attributed to such factors. “Data quality” or “quality” as used herein refers to the degree of noise in data. In some embodiments, data quality refers to the degree of noise in the image or video data recorded by a monitoring system, an image system, an image, optical, photographic, or video sensor, a processor, and/or any other device configured to capture image or video data.

“Continuous video feed” as used herein refers to both an uninterrupted video feed, and a video feed that is captured in sequence, but which may include periodic interruptions. In some embodiments, a continuous video feed is comprised of real time video data of any event occurring within or to the growth environment. In some further embodiments, a continuous video feed is comprised of continuous data.

“Image system” or “imaging system” as used herein refers to a camera, photographic system, and/or any image or video sensing system that can capture still and/or moving imagery. In some embodiments, the imagery can be in digital form, and/or can be captured using, e.g., stereo image sensors, multi-spectral sensors, three-dimensional image sensors, Time of Flight light sensors, thermal image sensors, or any other image sensing device. For example, digital images and/or digital videos can be transmitted to a computer processor and can be used as data or information by a software system that can be driven by an AI model. In some embodiments, “data” can refer to image or video sensing information captured by a camera, photographic system, and/or any image or video sensing system that can capture static and/or moving imagery. In some further embodiments, data is captured in the form of pixels, electromagnetic wavelength, frequency, amplitude and/or intensity, acoustic wavelength, frequency, amplitude, intensity, and/or saturation, thermal energy or saturation, chemical profile(s) (e.g., of the liquid mist and/or the gaseous content of the growth environment), barometric pressure, spatial resolution, dynamic range, frame rate/temporal resolution, sensitivity (ISO), electromagnetic, gamma correction, and any other form of information that can be captured by an image sensor. In some further embodiments, image or video data can capture optical, thermal, visual, infrared, near-infrared, acoustic, x-ray, chemical, electromagnetic wavelength, frequency, amplitude and/or intensity, and any other type of imaging information. In some further embodiments, data can refer to any information captured by, received by, loaded or read into, or output by the machine learning tools as disclosed herein.

“Feedback loop” as used herein refers to a process in which the output of a system is informed by its input, influencing subsequent outputs. In some embodiments, the output of a system is used as input to the same system, thereby creating an informational loop. In some embodiments, the feedback loop can be a positive feedback loop. For example, the output of a system can amplify or reinforce the input of a system, leading to a self-reinforcing cycle of a desired output. In some embodiments, the feedback loop is a negative feedback loop. For example, the output of a system can dampen or counteract the input of a system, thereby maintaining a desired stasis or equilibrium. In some embodiments, a mechanical or electronic system can automatically regulate the system. For example, mycelium growth can be maintained at a desired state or set point, e.g., resulting in a desired mature mycelium material. In some further embodiments, a feedback loop can operate with little or no human intervention. Human interaction may be involved as part of training machine learning tools/models for more dynamic systems. A nonlimiting example of a feedback loop in the context of optimizing mycelium growth can include using monitors or sensors to track environmental conditions and/or mycelial morphology, and automatically adjusting environmental conditions to achieve a desired mycelial product or end result.

“Deviant morphology” as used herein refers to an undesired mycelium morphology. In some embodiments, deviant morphology can be morphology that departs from desired or expected morphology. For example, a desired morphology can be one that includes predetermined aesthetic properties, physical properties, hepatic properties, flavor properties, color properties, or any other desired property of a mycelium. For example, a desired morphology can have a desired tensile strength, a desired flavor profile, a desired texture, a desired density, etc. In some further embodiments, a deviant morphology can be different from a desired morphology because the physical characteristics of the deviant morphology are unsuitable for further growth or use. For example, a deviant morphology can be unsuitable or undesired as a product or intermediate product, or a deviant morphology can be unsuitable or undesired because it has not yet met its targeted or expected growth or growth trajectory for the time it has been growing in a growth environment. In some further embodiments, deviant morphology can be “permanent” or “temporary.” Permanent deviant morphology can comprise morphological properties that make a mycelium entirely unsuitable or undesired for use, and/or unamenable to repair or modification through changes in growth conditions. For example, permanent deviant morphology can comprise substantially non-contiguous growth areas, morphological malformations caused by contamination events (e.g., from other fungal strains or other biological organisms), or any combination thereof. Temporary deviant morphology can comprise morphological properties that can relate to mycelium, or part thereof, immaturity, and/or delayed growth. Such temporary deviant morphology may be mitigated within a suitable growth timeline by changes to environmental conditions. For example, temporary deviant morphology can include repairable immature growth or development at particular stage of mycelium growth within a growth timeline. Such immature growth or development can be the result of, e.g., inconsistent application of nutrients in either the mist or substrate, or heterogeneous environmental conditions. For example, heterogenous growth conditions can include uneven mist levels across portions of the growth environment.

“Homogeneous” as used herein refers to the topology of growth of the aerial mycelium. In some embodiments, aerial mycelium is morphologically composed of variably expressed structures (e.g., bulbous structures) with varying degrees of diffusion within and between one another, and in height, with respect to each other. This may be referred to more generally as the “topology of growth,” “growth topology” or “surface topology.” The variable and eccentric expression of bulbous features and variable tissue density within and between bulbous features represents a challenge for example, in textiles applications. For example, tensile failure can selectively occur when morphological “bulb” forms become too discrete, due to a lack of cross-linking at the intersections between these forms, which can lead to variable failure modes and reduced physical strength. Conversely, increased homogeneity can increase tensile strength, for example, by increasing cross-linking.

“Lifecycle” as used herein refers to the developmental stages that fungi undergo, encompassing both sexual and asexual reproduction. For example, this process can involve distinct phases such as spore germination, hyphal extension, mycelial vegetative growth, formation of aerial mycelium/a, and/or the formation of specialized structures for reproduction.

“Solid-state fermentation” or “solid-state substrate fermentation” as used herein refers to a process wherein one or more organisms are grown on a solid substrate. For example, this process can involve a mycelium growing between and/or within the empty spaces of the solid growth matrix or substrate, thereby providing a solid physical support for growth. “Liquid-state fermentation” or “liquid-state substrate fermentation” as used herein refers to a process wherein one or more organisms are grown on a liquid substrate. For example, this process can involve a mycelium growing within or on the surface of a liquid growth matrix, liquid substrate, or nutrient broth.

### DETAILED DESCRIPTION

The figures and the following description relate to various example embodiments by way of illustration only. It should be noted that from the following discussion, example embodiments of the structures, methods, systems, and associated models disclosed herein will be readily recognized as viable alternatives that may be employed without departing from the principles of what is claimed. Reference will now be made in detail to several embodiments, examples of which are illustrated in the accompanying figures. It is noted that wherever practicable, similar or like reference numbers may be used in the figures and may indicate similar or like functionality. The figures depict embodiments of the disclosed methods or systems (with select equipment for purposes of illustration) and one skilled in the art will readily appreciate from the following description that various example embodiments of the structures, systems, methods and equipment described herein may be employed without departing from the principles described herein.

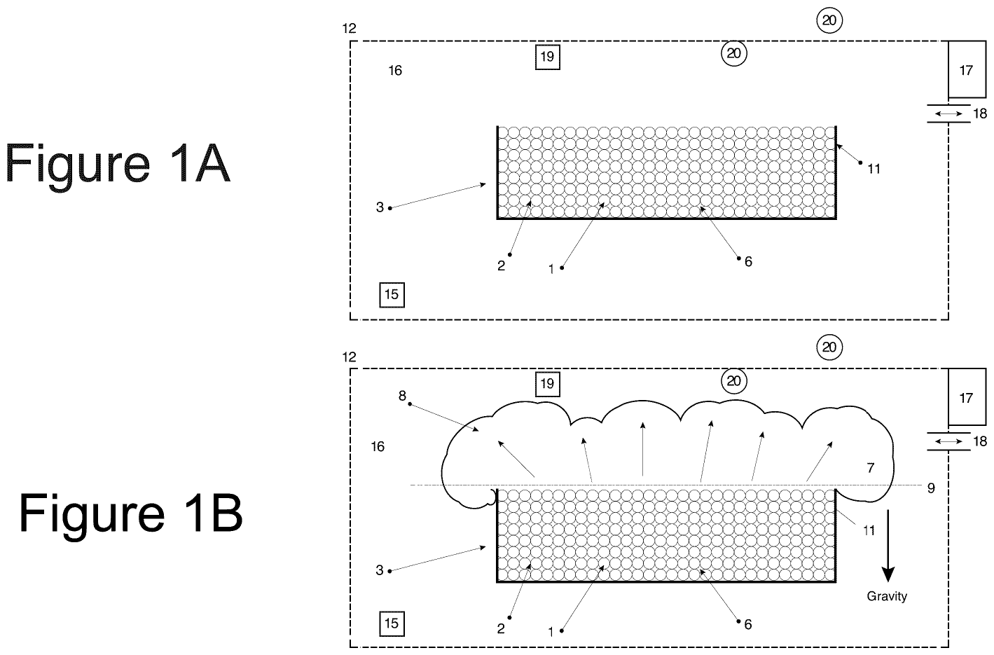

FIG. 1A illustrates an embodiment of a growth matrix 3 suitable to support extra-particle mycelial growth, such as extra-particle aerial mycelial growth. The growth matrix 3 is shown as circles. In some embodiments, the growth matrix 3 can be contained within a tray 11 with a bottom and side walls as shown. The growth matrix 3 can include a growth medium and a fungus. For example, the growth matrix 3 can comprise growth media 2, substrate 1, and colonized (or pre-colonized) substrate 6, to support growth therefrom.

The growth matrix 3 can be exposed to environmental conditions imparted by environmental controls 15, contained either within the growth environment 16 or outside a growth environment 16 (the growth environment shown in dashed lines) contained within a growth environment system 12. The growth environment 16 may be a relatively small growth chamber, such as a benchtop bioreactor in which a relatively small amount of aerial mycelial material is grown, or a relatively large growth room, in which numerous large portions of aerial mycelial material is grown, such as in extended panels. One or more environmental conditions can be monitored within the growth environment via environmental condition monitors or sensors (aka environmental sensors) 19, which can either directly, or through a processor 17, communicate the need for changes in environmental conditions within the growth environment 16 in response to unsatisfactory condition changes, or timed condition changes to environmental controllers 15 (located either inside or outside the growth environment). For example, implementing condition change that affects the growth from the growth matrix for desirable end results. For example, a processor 17 and environmental condition controllers (aka controls) 15 can be provided with information from the environmental sensors 19, leading the environmental sensor 19 to implement environmental changes to control oxygen (O₂) content, carbon dioxide content (CO₂), other gaseous content, atmospheric pressure, temperature, humidity, misting levels, misting liquid composition, misting direction, airflow direction and rate, light, and/or other environmental conditions to, from, and/or within the growth environment 16. For example, such changes can be achieved through an interface 18, or a location within the growth environment 16. Environmental controls 15 may include HVAC controls/inputs, heating or cooling elements, misting apparatus, lighting apparatus, humidifying apparatus, nutritional addition apparatus, contaminant control apparatus, and such. Such changes in environmental conditions by the environmental controls 15 may include not only the periodic turning on or off of environmental systems once a desired condition is attained, but also the directing or redirecting of certain condition changes to certain locations within a growth environment, such as for example, the redirecting of mist nozzles or airflow in a growth environment to place aerial mist at concerning locations where it is deemed lacking or would be most useful for improved growth, based on the results of data (and/or comparisons with either system data for other locations in the growth environment, or previously referenced data for earlier growth runs, demonstrating normal trajectories for growth of mycelium at that concerning location, and for that time period in the growth timeline). Other potential environmental condition controllers 15 may be used to introduce nutritional supplements or water into the system if deemed necessary or beneficial, such as to the growth matrix. The environmental condition sensors 19 may be traditional sensors such as thermostats, humidity detection apparatus, airflow velocity measurement devices, airflow directional measurement devices, gas detection devices, atmospheric pressure detection devices, light measurement devices, airborne liquid detection devices, contaminant detection devices, or nontraditional sensors such as infrared or heat imaging sensors.

In certain embodiments, the monitors and sensors may include image monitoring systems (e.g., optical, thermal, visual, infrared, near-infrared, acoustic, x-ray, chemical, and any other type of imaging), alternatively, visual monitoring devices, for example, image systems 20 (e.g., optical sensors or traditional cameras, including still image or video cameras). Image systems may include the following equipment: photodiodes and phototransistors, phototubes, photovoltaic cells, photonic sensors, fiber optic sensors, optical encoders, fluorescence sensors, reflective optical sensors, absorption spectroscopy sensors, interferometric sensors, lidar sensors, optical gas sensors, digital image sensors, mirrorless image sensors, digital single-lens reflex (DSLR) image sensors, action image sensors, compact image sensors, bridge image sensors, medium format image sensors, instant image sensors, 360-degree image sensors, web cameras, CCTV image sensors, thermal image sensors, trail image sensors, drone image sensors, film image sensors, smartphone image sensors, although any other suitable equipment may equally be applied. Such condition monitors 19 and image systems 20 may be singular in number or multiple, and may be placed at locations that are centralized for unobstructed monitoring such as in a central aisle, or throughout the growth environment 16 at locations such that essentially a monitoring array is present to communicate a representatively accurate sampling of conditions or growth levels throughout the entire growth environment from select locations. In certain embodiments, only image systems 20 may be present in single or multiple locations, rather than multiple types of environmental sensors within the growth environment 16, and changes in environmental conditions by environmental condition controllers 15 may be based solely on visual images (and comparisons with either other location images, or historical images for successful, similarly aged mycelial growth at that imaged location).

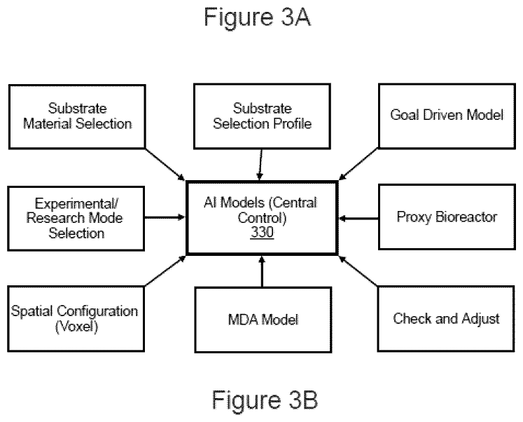

Such image systems 20 may be placed within the growth environment 16, or external to the growth environment 16, such as outside a window adjacent the growth environment 16, with ideally clear unobstructed view of the activities within the growth environment 16. Whether placed inside or outside the growth environment 16, such image systems 20 in one embodiment, desirably have the ability to address noise of captured images in the growth environment 16 or through a transparent surface (making up a window of the growth environment) and transmitted to a processor 17 (for evaluation by the processor and associated operational AI models, as described further below). The processor 17 may be singular or numerous as desired, and may be connected to a network 310 and other operational features (as described further in FIG. 3A) including multiple monitors, sensors (environmental condition sensors), image systems (optical sensors) 20, optional alert and reporting systems 318, optional operational safety management systems 327, and AI model-associated data management systems 340.

In some embodiments, the growth matrix 3 is implemented without tray 11 (e.g., on another growth support structure, such as a planar support structure without side (or even bottom) walls, such as a mycological growth web, net, shelf, rack, or other supporting system.

FIG. 1B illustrates an embodiment of extra-particle aerial mycelial growth 8 from the growth matrix 3 of FIG. 1A. For example, the growth can occur when the growth matrix 3 from FIG. 1A is incubated or otherwise processed within a growth environment 16 under growth conditions suitable for the desired properties of the extra-particle aerial mycelium growth 8 in FIG. 1B. For example, in some embodiments, the environmental condition(s) within the growth environment can be controlled to induce substantially homogeneous extra-particle aerial mycelial growth from the growth matrix.

The extra-particle aerial mycelium growth can extend away from and outward from a surface of the growth matrix to form an aerial mycelium 7 as shown. Appropriate growth conditions of the growth matrix 3 in FIG. 1A result in extra-particle aerial mycelium growth initiating across the exposed surface of the growth matrix. Next, extra-particle aerial mycelium growth continues to expand, forming a volume of extra-particle mycelial growth 8 as shown in FIG. 1B. The volume of growth can be generally contiguous, in that little or no gaps or holes in the extra-particle mycelial growth 8 occurs. The extra-particle aerial mycelium growth 8 can be grown to various heights. In some embodiments, the growth is about 3-4 inches high above the growth matrix 3. This can be achieved, for example, in up to about two weeks of growth, during which time the extra-particle aerial mycelium growth can become a mature mycelium. It will be understood that although the extra-particle aerial mycelium growth has some amount of irregularity to its upper surface topology as shown, the drawings are not to scale, and the top surface can be relatively flat when viewed from a certain distance. The extra-particle aerial mycelium growth 8 can be separated from the growth matrix at a position 9, such that no growth matrix is transferred in the separation.

As noted, in some embodiments, the growth can be implemented on a mycological growth web, for example, without the tray 11 shown. The growth web can include the growth matrix and the extra-particle aerial mycelial growth (e.g., without a tray 11). The growth web can include any suitable support structure to support the growth matrix 3 and the extra-particle aerial mycelium growth 8, such as a net-like structure, or other perforated material. The web can be a standard size, such as a 63″W×38′L, 63″W×98′L or any of many other web configurations. Other sizes can be implemented, including lengths up to 90, 100 feet, or more. The net can comprise one or more layers of a perforated or nonperforated material, or combinations thereof, such as a plastic, nylon (e.g., nylon weave), or any other flexible, suitable material or multiple layers of material for growing extra-particle aerial mycelium growth 8 from a growth matrix 3. The web can extend in length from right to left in the orientation shown in FIG. 1B. As illustrated, the growth 8 demonstrates negative gravitropism, although positive gravitropism is also contemplated to be within the scope of the inventive methods and systems.

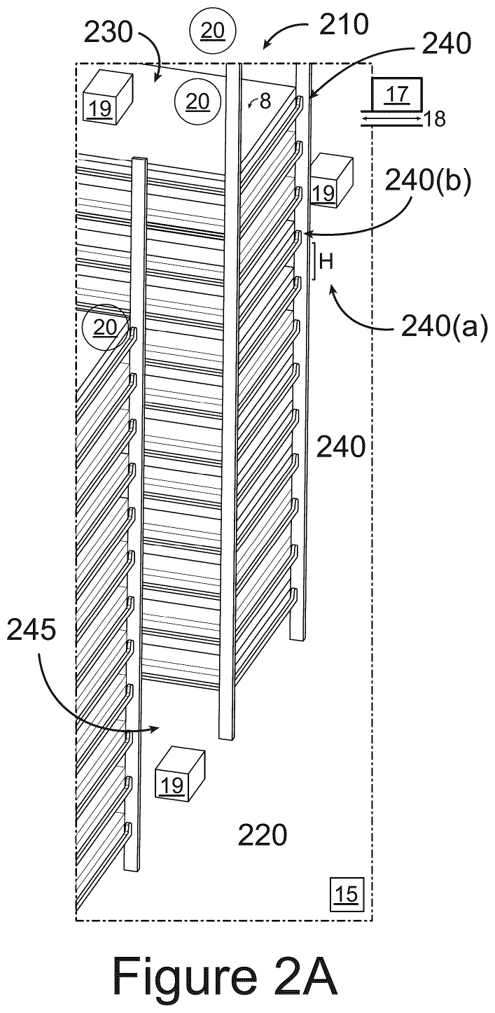

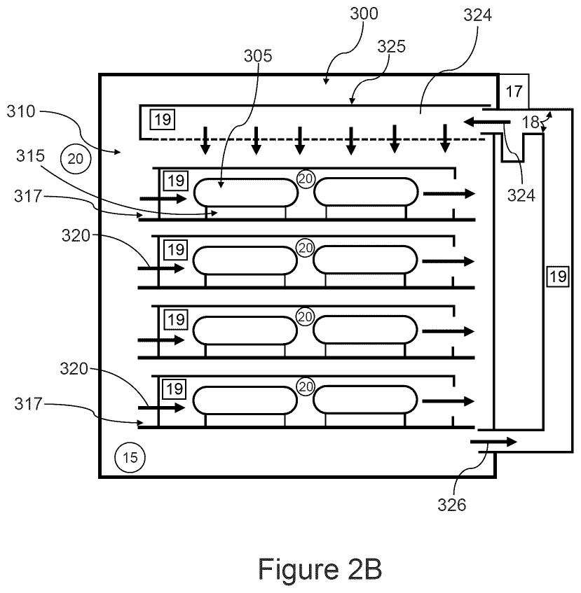

FIG. 2A illustrates an embodiment of an isometric view of a growth environment system 210 for cultivating aerial mycelia within a growth environment 220, such as in a larger growth room or chamber. It should be understood that each of the methods and systems described herein can be applied to growth environments of a significant size. The growth environment 220 can be configured to grow mycelium from a growth matrix positioned therewithin. For example, system 210 and growth environment 220 contained therein can be implemented to facilitate the mycelial growth described above with reference to FIGS. 1A-1B and can include similar components and functionality.

The growth environment 220 can include one or more shelves 240 (e.g., vertically configured shelves), on which a growth matrix can be positioned, and from which extra-particle mycelial growth can extend. The growth matrix can be positioned directly on a shelf, or with an intervening growth support structure, such as a growth web, net, or bed. One or more racks (or shelving units) 230, such as the two racks shown separated by an aisle 245, can include a plurality of the shelves 240 (e.g., stacked vertically), positioned within the growth environment 220. The environment can include various numbers of racks, which can include various numbers of shelves 240, and can be of various dimensions. For example, two or more racks 230 can form two sets of shelves 240, wherein each shelf in each set can be positioned at approximately the same height as a corresponding shelf in the other set of shelves. A shelf can be sized and configured to support a web, net, bed or other growth support structure, such as those described above with reference to FIGS. 1A and 1B, or even other growth support structures. In some embodiments, the racks include 12 shelves and are 12-16 feet high. The shelves 240 can move vertically to compensate for spacing shelves 240 at different heights. The shelves 240 can be spaced apart from each other (e.g., vertically) a spacing height H that is sufficient to allow for the height of the desired extra-particle aerial mycelial growth combined with the height of the underlying growth matrix, sufficient clearance for airflow, mist flow, processing, and handling. It will be understood that air, air content and airflow as used herein can refer to various compositions of various gases, and flow of those gases suitable for the products and methods described herein and should not otherwise be limited to a particular composition or ratio of gases. Additionally, some embodiments herein that are described as being implemented with respect to “water” may be similarly implemented with other liquids.

The spacing height H is defined as the distance between the same two corresponding points on two adjacent shelves 240. For example, the spacing height can be defined as the distance from the top surface of a first lower shelf 240 (a) to the corresponding top surface of an adjacent upper shelf 240(b). In some embodiments, the spacing height can be in a range between about 200 mm to about 530 mm, or between about 225 mm to about 490 mm, or between about 250 mm to about 450 mm. In some embodiments, the spacing height can be less than 530 mm, less than 490 mm, or less than 450 mm. In some embodiments, the spacing height can be about 350 mm. These spacing heights can be advantageous because the nature of the mycelial growth herein requires less spacing, and thus can allow for an increased number of shelves and higher output than conventional mushroom cultivation. Such a growth environment 220 can include one or more environmental condition controls 15, one or more environmental condition sensors 19, and one or more image systems 20 (optical sensors such as image sensors), each designed to specifically encompass/monitor either a desired portion of the growth environment or the entire growth environment 220. Such sensors, such as the image systems 20 may be positioned within the growth environment 220 or outside such environment, such as adjacent a glass window in a wall defining the growth environment.

FIG. 2B illustrates an embodiment of a system 300 for creating a growth environment 310 for growing aerial mycelium 305, such as on a commercial scale. Another example of a system 300 for creating a growth environment 310 for growing aerial mycelium 305 on a commercial scale can be seen in FIG. 2B, which shows a schematic view of a growth environment or growth chamber 310 having a series of trays 315, or beds on racks on shelves 317 contained in the controlled environment in which airflow 320 is directed horizontally across each of the trays, containers, or beds after entering the chamber through a manifold 325. In the illustrated schematic, airflow enters the manifold at an entrance 324, travels through the manifold and is directed to one side of shelves 317 where it is allowed to enter discrete spaces 320 and pass horizontally over the surfaces of growth matrix and/or aerial mycelium growing therefrom 305. The direction of airflow is illustrated throughout the schematic by the directional arrows. It may exit at 326 and be reintroduced and redirected to target locations across the growing aerial mycelium and/or substrate or matrix.

Environmental condition monitors/sensors 19 are placed about the growth environment 310. As with prior system embodiments, a processor 17 is present to communicate with one or more optional environmental condition monitors 19, one or more environmental condition controllers 15, and one or more image systems 20 to control conditions within the growth chamber 310 in response to observations from said monitors, 19, 20. The numbers and placement of the monitors may be few or numerous, based on the homogeneity of conditions throughout the growth chamber, and/or the capabilities of the chosen sensors/monitors. In one embodiment, the range of monitors include both image systems 20 to periodically monitor the actual growth (such as for example current growth condition and/or calculated growth rate based on a series of compared images and algorithmic calculations) of aerial mycelium off of the substrate or growth matrix, and environmental condition monitors 19, with both types of monitors communicating with a processor or a network of processors as may be desirable (which are either centralized or dispersed and with either centralized or dispersed operational models associated therewith).

The various sensors in the growth environment 310 communicate/transmit data/images to the one or more processor(s) which runs one or more associated evaluative models (based on previously loaded data management systems). Based on the one or more various models, adjustments as made as needed to the environmental conditions via the environmental condition controller(s) 15 in the growth environment 310 in order to achieve a targeted aerial mycelial growth level and type, for the time period desired, or alternatively, to make up for immature growth in particular locations within the growth environment 310, or still alternatively, to identify and properly address permanently deviant aerial mycelium morphology which may be present in the growth environment 310.

FIG. 3A illustrates just one example of aerial mycelium growth system architecture 350 for organizing an aerial mycelium growth environment according to one embodiment of the invention. The system architecture 350 includes a network 310, in communication with one or more optical sensors 312, one or more optional environmental sensors 314, controller elements of an environmental control system 316, optional alert and reporting systems 318, to communicate select alerts to human managers of the system architecture 350 and log status of conditions generating alerts, optional operational safety management systems 327 to effectuate steps to address safety issues that may occur within the system architecture 350, record entries of specific details and prepare and transmit related communications. Environmental control system 316 can be configured for implementing action steps to alter environmental conditions relating to for example, HVAC (airflow, airflow velocity, airflow rate, airflow direction, and airflow heating and/or cooling), gas content (such as by off-gassing if necessary undesirable gases), mist deposition (such as to initiate airborne liquid mist, regulate such mist levels, composition and/or direction, discontinue such mist, redirect such mist, either in periods by timing, or as needed, to provide for example, increased moisture or nutrient levels), light levels, relative humidity, substrate composition (such as moisture and nutritional levels) and such. For example, one action step can include one or more compositions of mist from one or more mist sources (e.g., various mist-containing, nutrient formulations available to one or multiple locations within a growth environment. In some embodiments, the action steps to modify one or more environmental condition can be implemented by at least one of a temperature control device, a mist control device, a relative humidity control device, an atmospheric pressure control device, and an atmospheric gas control device, an airflow control device, or any other device that relates to HVAC, gas content, mist deposition (including to initiate airborne liquid mist, to regulate mist levels, composition and/or direction, to discontinue mist, to redirect mist), light levels, relative humidity, substrate composition or any other environmental condition. For example, a mist control device can be configured to control misting levels, misting liquid composition, misting direction, or any other misting action. Also connected to the network 310 are one or more AI models 330 (such as on processors) and associated data management system(s) 340 that may be relied on by said AI models for continuous learning, with said data management systems being regularly and continuously updated, such as for example with growth progress information from experimental and commercial growth runs, globally available networked growth runs, historical data, publicly available data on relevant aerial mycelium equipment specifications, substrate (flora) material local availability, and other such information.

The various monitors, sensors, or devices positioned in and around the growth environment may include for example, one or more optical sensors 312 (such as RGB image sensors, photographic, or videographic sensors), and environmental sensors 314 (such as temperature sensors, humidity sensors, infrared image sensors, thermal image sensors, air monitors, mist visibility monitors (including monitoring mist liquid compositions and/or direction), light monitors, gas monitors etc.) A set of sensors may be distributed throughout the growth environment to sense data at various locations throughout the growth environment in an array or lattice configuration, such as one or more for each shelf or rack or bed, or centrally located sensors, strategically placed in locations that are representative of conditions throughout the whole growth environment. The optional operational safety management system 327 may maintain the general operational safety program for the growth environment. In some embodiments, the operational safety management system 327 keeps records related to operational safety for the growth environment. The optional alert and reporting system 318 monitors, logs, and reports the operations of the system elements, the one or more types of sensors, and the software running in the system (such as in the processor(s) 17 and network 310), the AI models 330 and the data management systems 340.

FIG. 3B illustrates types of AI models 330 that may be utilized by the system in accordance with the invention, and includes at least substrate selection models, goal driven models, proxy bioreactor models, check and adjust models, and spatial configuration models, each of which are described in further detail below. In some embodiments, the AI models 330 can be descriptive, calculation-based, or both. For example, hyphae can be distinguished from other surrounding features based on an analysis of pixel color in visual image data, or from infrared sensor data describing visual contrasts in grayscale. In some embodiments, the AI models 330 involve calculation-based steps. For example, sensor data can be used to infer, e.g., growth rate from pixel changes over time, mist velocity based on pixel changes, 3D point clouds to analyze growth rate, etc. In addition to the pixel coordinates in two dimensions, the distance to the image sensor can add a third dimension of analysis. In some further embodiments, the AI models 330 can allow inferences to be made even where there is missing data. For example, a neural network AI model that has been trained with images of desired mycelium attributes at specified time points can be used to infer mycelium properties at stages in the growth cycle for which there are no data (e.g., by implementing Kalman filters). Such images can be used, e.g., to adjust environmental conditions in the growth environment in response to changes in environmental conditions and/or organismal responses.

In some further embodiments, the AI models 330 can use sensor data to build a “model image.” For example, during the training phase, images features (segments, edges, etc.) can remain undescribed or uncategorized, and can be used as input information for the neural network. The neural network can decompose images into discrete units that can be used to predict and to control the growth environment. For example, a neural network can use images of mycelium to create “attention maps” that can be used to determine whether a mycelium is homogeneous without requiring the provision of images that have been analyzed and determined by a human to represent homogenous mycelium.

In some further embodiments, the AI models 330 can use inference-based methods to control a growth environment based on probability calculations. For example, an AI model can analyze current and/or a time course data of growth environment conditions to probabilistically determine how growing mycelium should appear at a particular stage of a growth run.

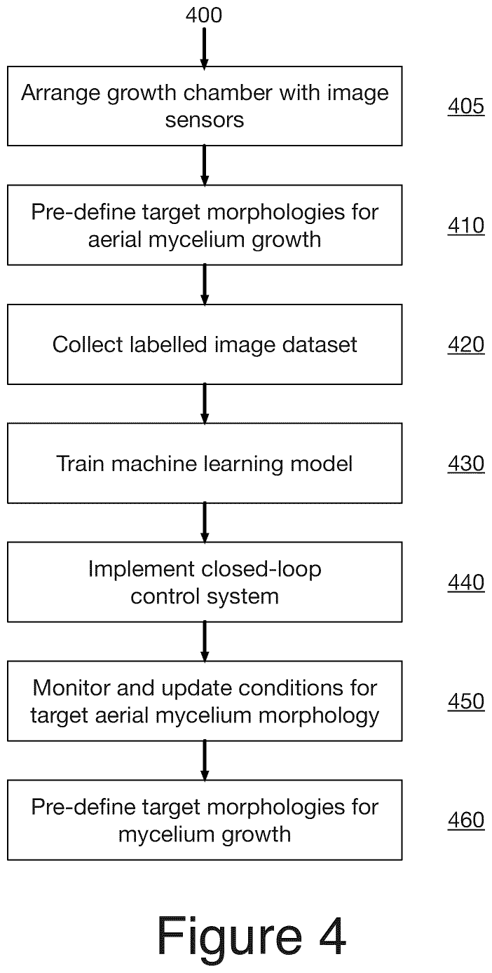

FIG. 4 illustrates an embodiment of a method utilizing machine learning tools/models to effectuate desired growth of aerial mycelium 400 in a growth environment (e.g., growth chamber). The machine learning tools may be first arranged with one or more image systems (e.g., image sensors) 405 in a growth environment (in addition to desired environment condition monitors, environmental condition control equipment and various networking, processing, data management, and modeling programming previously described). A predefined target morphology of aerial mycelium may be provided to the system 410. As aerial mycelium is triggered to grow in the growth environment (by initiating the growth conditions), periodic images of the growing aerial mycelium may be collected by the image system(s) and labeled as an image dataset 420. Either concurrently or previously, a machine learning model may be trained to recognize the collected labeled dataset from the image system in order to understand whether such images represent a predefined target for expected growth morphology, such as by an associated time or not (that is, on an expected trajectory of growth or not by a particular timeframe within said growth run, and also whether said growing aerial mycelium is uniformly growing across the desired growth environment (if desired) 430). It is generally preferred that the growth of such aerial mycelium is desired to be uniform throughout a growth environment, but for in some embodiments, such growth may be intentionally staggered or varied throughout a growth environment in order to achieve multiple different growth targets (or goals), within the same growth environment. In response to consideration of such image dataset, the system responds to implement an appropriate change in condition as and where necessary, using the environmental condition controller (aka closed-loop control system) as appropriate 440, such that the system utilizes a feedback loop arrangement to check and adjust its growth conditions in one or more growth environment locations, based on real time visual observation collected in the image system image dataset. Such check and adjust (e.g., monitoring and update of conditions for at least target morphology and across multiple growth environment locations as desired) 450 occurs periodically throughout the programmed/expected growth period of the aerial mycelium. The periodic check and adjust may be continuous or staggered at set periods. During expected periods of rapid growth for example (such as during specific day periods during a growth timeline), such check and adjust may occur more frequently. As the system checks and adjusts, it may be programmed to also regularly refine its control system performance (either based on current and future run successes and failures, comparison with updated data from other sources, and/or additional human intervention and input) 460.

A more detailed example of the method illustrated in FIG. 4 includes the following specific steps. The following detailed method is provided as a single, non-limiting example of a check and adjust AI model.

### Example of Check and Adjust AI Model:

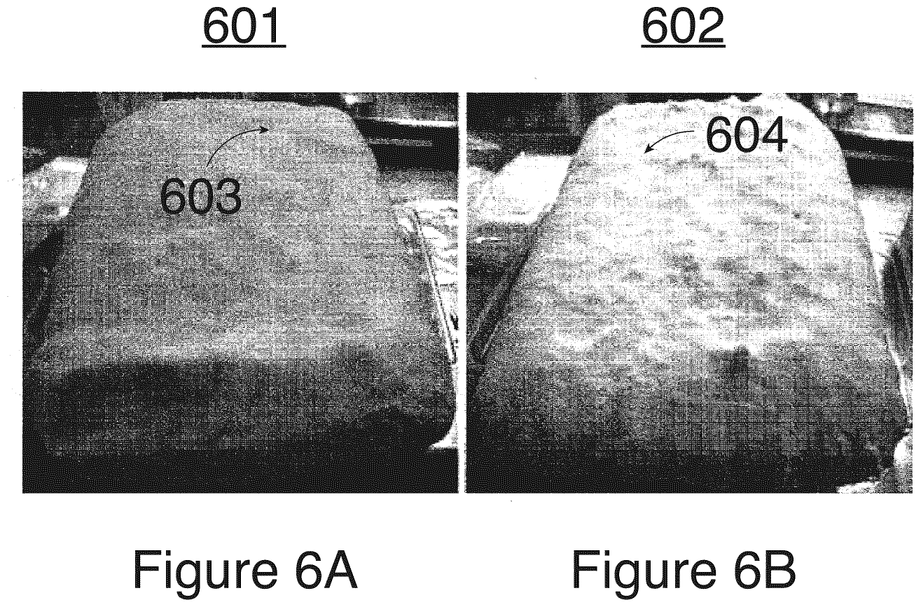

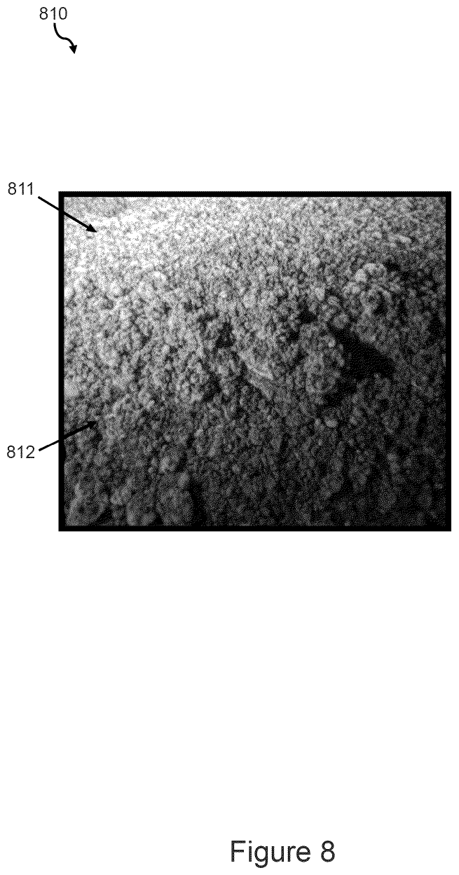

1. A growth environment, for example, including some of the features of FIGS. 1A, 2A, 3A is arranged with image systems (e.g., image sensors) positioned within either a centralized location above a growth matrix (contained in a bed, tray, rack etc.), to capture at least top, and alternatively top and side views of aerial mycelium growth on a growth matrix;
2. Target morphologies, including, for example, some of the features of FIGS. 6A, 6B, 7A, 7B, and 9, or historical image appearances for various aerial mycelium growth stages, including, for example, some of the features of FIG. 8, are pre-defined, based on the desired mycelium or mature mycelium morphology or physical attribute requirements;
3. A dataset (historical and current) is collected from the included image systems, of labeled images representing “acceptable” and “nonacceptable” growth patterns from previous internally-generated experiments, published documents in the relevant field, or expert knowledge;
4. A machine learning tool (such as one or more of the models described herein) is trained using the labeled image dataset to recognize and classify aerial mycelium growth patterns/morphology, both acceptable and non-acceptable. Such non-acceptable aerial mycelium growth patterns may include, for example, those patterns having unfavorable surface topographies, unfavorable physical features, such as deviant morphologies (deviant physical features, such as undesired texture, protrusions, color etc., perceived physical attributes), or irregularities based on time in growth period, or surface features previously associated with unacceptable physical attributes (like tensile strength, density, taste, or other). It is contemplated that multiple machine learning models could be functioning concurrently to integrate multiple imaging paradigms with each other for a more targeted environmental condition controller response;
5. A closed-loop control system that continuously captures images from the one or more image systems (e.g., image sensors), processes them using the trained machine learning model, and adjusts environmental growth conditions/parameters accordingly of the growth chamber through the associated environmental condition controller(s) (e.g., temperature, humidity, nutrient supply, mist levels, gaseous content etc.);
6. The aerial mycelium growth is monitored throughout the process, and conditions are updated via the environmental condition controller(s) to effectuate the target morphologies or appearances as needed, and so as to refine the control system's performance currently and for future growth runs.

In this fashion, the feedback-loop nature of the control system is driven by an image system and machine learning model.

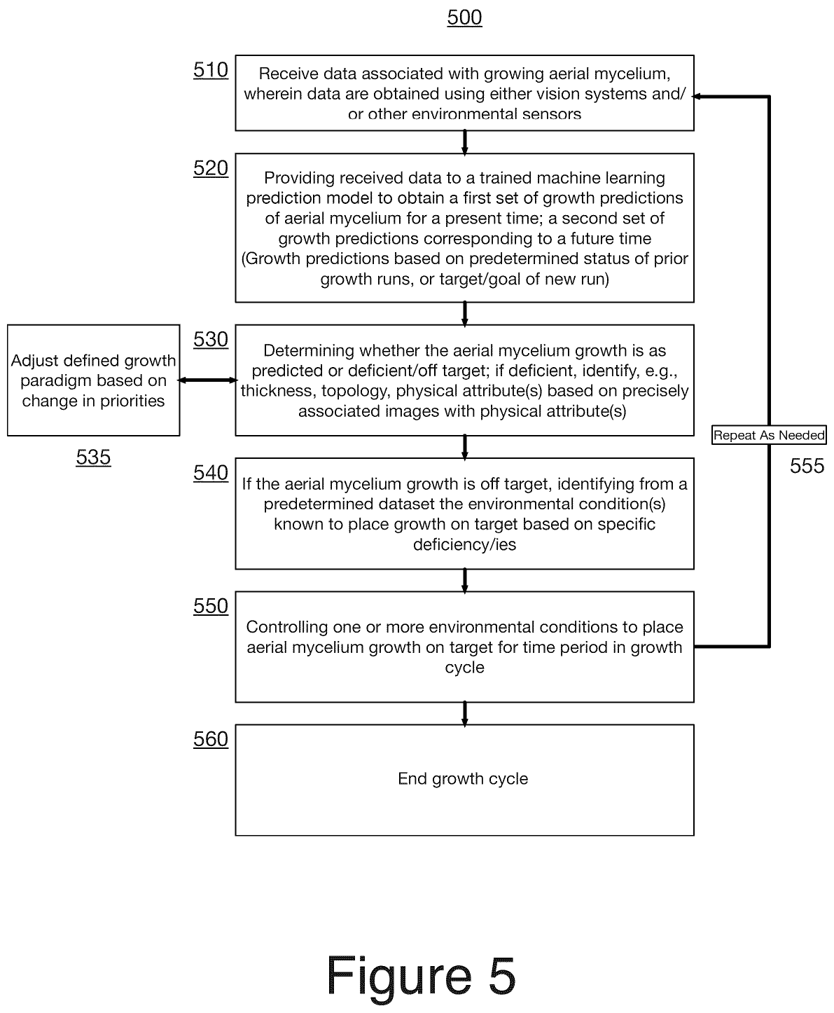

FIG. 5 illustrates an embodiment of a system or method 500 for growing aerial mycelium. In some embodiments, a growth environment with previously described controller and supporting equipment is provided, having either one or more environmental condition sensors, one or more image systems (optical sensors), or a combination thereof for monitoring the environmental conditions and/or morphology of growing mycelium growing in said growth environment, wherein data received from said sensors is received from said processors and interpreted by machine learning models 510. The machine learning tools/models have previously been trained with a prediction model to obtain a first set of growth predictions of aerial mycelium to a present time, and a second set of growth predictions corresponding to a future time (the growth predictions based on pre-determined status of prior growth runs or target goals for a new growth run) 520. The machine learning tools proceed to determine whether the aerial mycelium growth is as predicted or deficient in certain ways (off-target growth trajectory). If the growth is deficient, the model will identify the deficiency (such as for example, thickness, color, density, topography, or physical attribute based on previously associated image correlated with physical attributes) 530. If the aerial mycelium is off target for a given period of time in a growth timeline (by location if desired), identified by comparison with a predetermined image or sensor dataset, the relevant environmental condition or conditions correlated with curing the deficiency is identified 540. At this point in the model, if there is also a change desired in grown material priorities, and a grown aerial mycelium having different final properties/physical attributes is desired, such condition changes are implemented (with human intervention) to adjust/modify the desired growth outcome 535 with concurrent model adjustment to accommodate the new set of attributes being sought.

Assuming no change is desired in final grown aerial mycelium material priorities, environmental controllers in the system control one or more of the identified environmental growth conditions to place the aerial mycelium growth on target trajectory for the next appropriate monitoring period in the growth timeline (or growth cycle) 550. If following the adjustment of environmental conditions by the appropriately identified environmental controller(s), future monitoring reveals a continuing deficiency, the model reconsiders the new data for continuing or differing environmental condition changes using environmental controllers 555. If, however, the target aerial mycelium has been achieved (e.g., e.g., in the mature mycelium), the growth cycle (growth timeline) is ended.

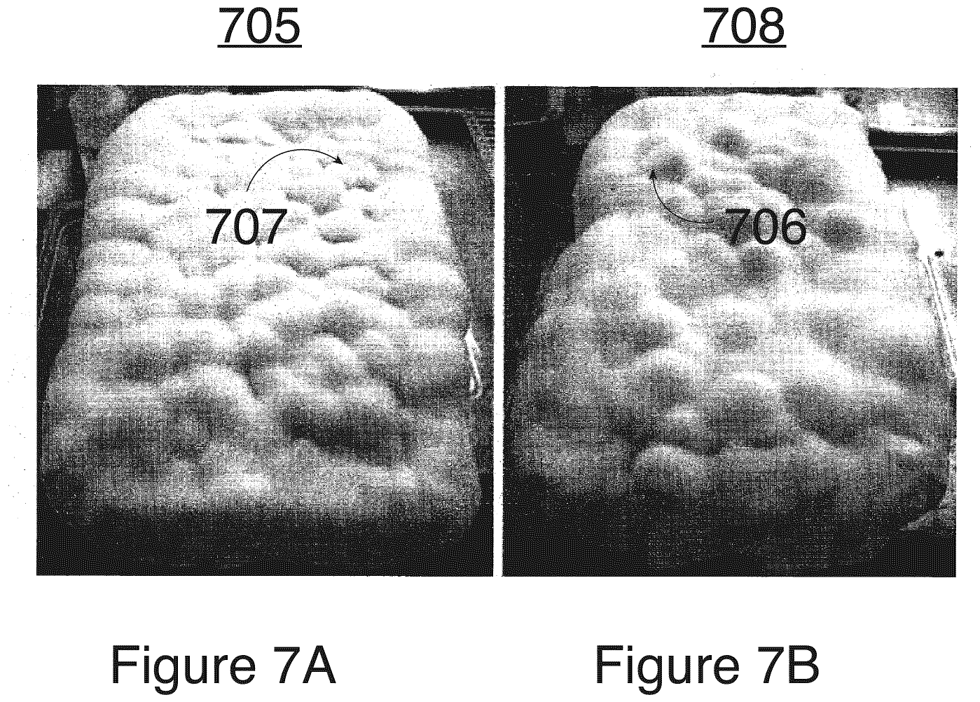

As shown in the photographic images of FIGS. 6A and 6B, exemplary fully grown panels 601, 602 of aerial mycelium are seen having relatively flatter, homogeneous surface topographies 603, 604 with relatively low levels of pronounced bulbous features. Such images are exemplary images of targeted trajectories to be utilized by models described herein. In comparison, as shown in photographic images of FIGS. 7A and 7B, exemplary panels 705 and 708 of almost fully grown aerial mycelium panels are seen 707, 706, having relatively highly pronounced bulbous surface features (especially when compared with the flatter surface features of panels 603 and 604). Such images may likewise be used as targeted trajectories based on growth timeline points, or final endpoints, or may also serve as examples of unsuccessful materials to avoid, if bulbous features and physical attributes associated therewith are deemed undesirable features of the mycelium.

In some embodiments, the image system described herein can capture images, of aerial mycelium, including those as illustrated by FIGS. 6A, 6B, 7A, 7B, 8, and 9. For example, an array of image sensors can be configured to capture a three-dimensional profile of the aerial mycelium. In some further embodiments, the array of image sensors can capture several variables including, but not limited to, area fraction, color/tonal value, variance of color/tonal value, mycelium geometry, or any other variable relevant to mycelium topology, geometry or morphology. Such variables can be further recorded at desired time intervals throughout the course of mycelium growth. In some embodiments, images can be used to identify mycelium features at instantaneous timepoints, including, but not limited to: growth height, growth area, surface roughness, volume and growth directional anisotropy from assembled two-dimensional images, geometric morphometry to derive number and geometry of bulbs/discrete morphological features and spatial clustering/autocorrelation of morphological features. In some further embodiments, the rate of change in any of the aforementioned or otherwise relevant to mycelium topology, geometry, or morphology, can be calculated. The calculated rate of change could be combined with other variables from the growth environment (e.g., mist levels, mist liquid composition, mist direction, temperature, airflow velocity, metabolic evolution via atmospheric gases and substrate temperature, substrate moisture content, substrate pH, and any other growth environment variable) to indirectly predict additional mycelium features (e.g., total mycelium mass, mycelium moisture content, and any other desired physical property of the mycelium) from which further data can be derived (e.g., mycelium wet/dry density, metabolic phase, anticipated endpoint timing and yield/morphological qualities with a probability of a given distance from the intended target, and any other variable relevant to mycelium production).

As seen in FIG. 8, early-stage growth of aerial mycelium on a growth bed 810 can be seen. In particular, white color-appearing aerial mycelium growth 811 is first starting to appear upon the surface of growth matrix 812 in the growth environment at a particular day in the growth timeline. AI models in accordance with the methods and systems of this invention would be trained to recognize such normal level of growth at such defined growth stage (in a growth timeline) under particular environmental conditions (for certain fungal organisms feeding on particular growth matrices) and use such image to determine deviancy from target physical attributes, and ultimate achievement of target morphology (and associated physical attributes).

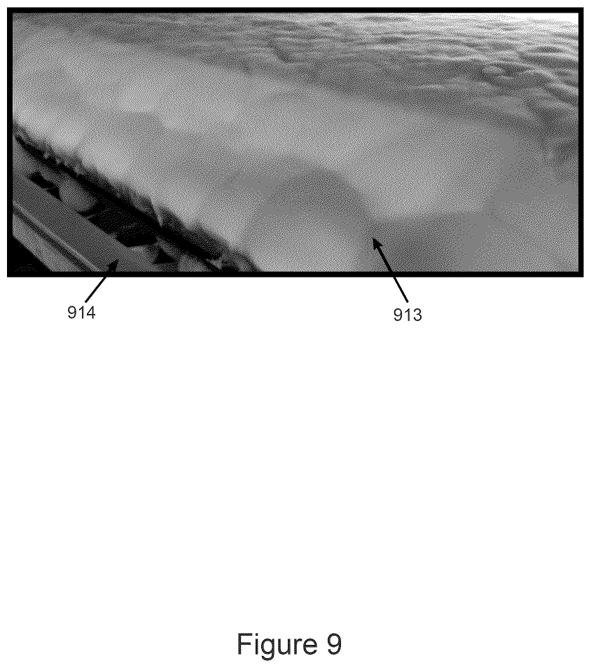

In contrast, in FIG. 9 thicker aerial mycelium growth 913 (e.g., in a mature mycelium) can be seen on growth beds (on racks 914), contained in a growth environment (e.g., relatively large growth chamber). As with FIG. 8, the image data from this later aerial mycelium growth stage would be used to consider the propriety of said growth condition at a particular stage in a growth timeline (but likely towards the end of the growth timeline). By comparing these images with predefined images accessible to models from continually updated data management systems, conditions can be adjusted to reach a desired outcome.

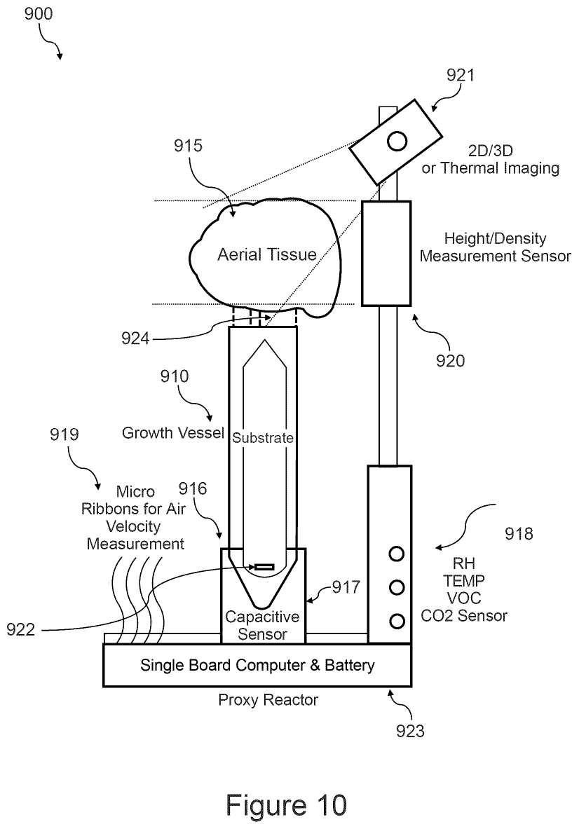

FIG. 10 illustrates an embodiment of a “proxy” bioreactor model, e.g., a modification of aerial mycelium growth environment hardware, to support expedited cost-saving AI modeling of unfamiliar growth environments. Modeling the thermodynamic activity within large volume growth environments is challenging, as it is difficult to build accurate models that can capture the interactions between growth condition variables, such as airflow, relative humidity, gas diffusion, and temperature, for the following reasons. The distribution of thermal energy in large growth environments varies at different locations and elevations inside the growth environment. Furthermore, the interaction between airflow, humidity, and gas diffusion varies based on the changes in the airflow pattern (e.g., laminar, turbulent, etc.). While state-of-the-art air velocity measurement tools (e.g., hot wire anemometer) can detect low speed airflow (e.g., e.g., less than 2 m/s), they do not operate accurately in high relative humidity, which condition is common in aerial mycelium growth environments. Furthermore, thermodynamic interaction between these components significantly varies based on the geometry of the environment (width, depth, height), the layout of the HVAC ducts, location of the heating/cooling coils, and the layout of shelves/racks used for supporting growth matrix and aerial mycelium tissue growth.

Hence, in certain circumstances, one computational AI model of a chamber may not easily translate to another geometry/layout, limiting the translation of knowledge across different growth environments.

Therefore, in a further example embodiment of the invention, a growth environment is equipped with “proxy” bioreactors, which, for the purposes of this disclosure, are discrete aerial mycelium growth vessels embedded in-line with the to-be-evaluated growth environment, such proxy reactors placed in-line with a growth environment bed or tray, that provide location-specific information that affect the aerial mycelium tissue growth. These discrete vessels function as proxies to their neighboring full-sized growth environment bed, tray or other growth matrix-containing/supporting surfaces (e.g., section of a bed), and capture condition variations at different locations and heights within the growth environment (e.g., chamber). Data collected from a network of these proxy reactors can then be used to characterize entire growth environments, and their yield, without prior knowledge of their layout or control systems. These preliminary research tools can provide meaningful data, without incurring the costs normally associated with full commercial scale operation of the entire growth environment, including its full loading of growth matrix beds and inoculum for example.

Such a proxy reactor network can standardize the characterization of any growth environment, and taken with the disclosed dynamic environmental condition models, can allow for the eventual growth of consistent aerial mycelium materials, despite equipment, conditions or availability, and available growth substrate variances.

One embodiment of an exemplary proxy reactor comprises individual growth vessels (multiple vessels in a network) that are distributed across a growth environment (e.g., such as for example a mycelium production facility or a current mushroom farm that may be reconfigured to produce aerial mycelium instead) at different shelf/rack locations and elevations. The proxy reactors may be placed directly on the beds, trays, or shelves where mycelium production either currently takes place or is desired to take place. Mycelium that is grown in the proxy reactor growth vessels experience the exact environmental conditions as would the mycelium of the neighboring beds or trays of growth matrix, and therefore become representative of the successes or failures of mycelium that grow or are planned to be grown in the bed or tray adjacent them.

By precisely measuring the environmental conditions that affect the growth in the proxy reactor growth vessel, the proxy reactors provide accurate representation of the thermodynamic activity within that location. As they directly measure the growth of aerial mycelium tissue, they use aerial mycelium itself as a proxy-type sensor, to capture the effects of the environmental conditions that act on them.

One embodiment of a proxy reactor 900, as seen in the representational image of FIG. 10, includes a growth vessel 910 that is used to grow aerial mycelium tissue. In some embodiments of the proxy reactor 900, the growth vessel 910 can be cylindrical as shown (e.g., 50 ml falcon tube) or alternatively, rectangular or other shape, in order to accommodate different configurations. Multiple proxy reactors 900 with different geometries and equipment details can be tiled together to create larger volume growth vessels. In the illustrated embodiment of FIG. 10, the growth vessel 910 is attached to a stand 916 which holds a variety of sensors 917, 918, 919, 920, and imaging equipment 921. The imaging equipment 921 (e.g., optical, UV, thermal or 3D image sensors) directly observes the aerial mycelium tissue 915 growth over the growth vessel 910. The bottom of the growth vessel 910 consists of a removable cap 922 where a capacitance sensor 917 is attached to measure the amount of liquid displacement in the growth vessel 910. The proxy reactor stand includes a holder (not shown) to stabilize the growth vessel 910, The proxy reactor 900 includes visual indicators 919 (e.g., micro ribbons) to capture airflow activity with image analysis, and substrate level indicators 924 are also positioned along the side wall of the growth vessel 910. Other airflow and mist sensors can also be employed as part of the proxy reactor system.

The proxy reactor 900 includes additional sensors 918 to measure various growth environment conditions, such as for example, the local relative humidity, temperature, CO₂ and Volatile Organic Compound (VOC). The illustrated proxy reactors 900 include a single board computer proxy 923 that operates with batteries. It transmits data in one embodiment, wirelessly (e.g., Wi-Fi®, BLUETOOTH®) to a data collection hub (not shown), which then, in one embodiment, sends them to a database on the cloud. It should be appreciated that the illustrated proxy reactor 900 may include all or only a selection of the components described above, based on the needs of the evaluation being considered.

A proxy reactor network may include in one embodiment, at least physical twin proxy reactors 900 and digital proxy twins represented in software. Physical proxy reactors 900 transmit airflow, relative humidity, temperature, tissue height, CO₂, VOC data (or other desired environmental conditions) in real-time to a database (not shown). The data from each proxy reactor unit 900 is used to build a model of the thermodynamic activity inside the entire growth environment (e.g., chamber). The proxy reactor digital twin model can also visualize the aerial growth of mycelium tissue using computer graphics and diffusion models.

In one embodiment, as each sensor (such as those of 917-921) reports the local conditions on a bed, shelf, rack or other growth matrix support, the simulation model can interpolate the environmental conditions between each sensor and create a heatmap-style representation of substrate, tissue, and environmental conditions across the growth environment. From a grid of such proxy reactors 900 one can interpolate the conditions in-between proxy reactor physical locations.

In some embodiments, a proxy reactor can be configured with predetermined data, including substrate depth, substrate weight, fungal species and/or strain, the period of time for which mycelium has been growing in the growth environment prior to the addition of a proxy reactor, and additional nutrients in the form of either mist or substrate. These data can be integrated within the machine learning model so as to determine which variables most impact end performance. In some embodiments, the proxy reactors can serve as proxies for the performance of the whole growth environment, with data collected therefrom usable as a proxy dataset of the whole growth environment method or system.

In some further embodiments, a proxy reactor also includes sensors to monitor the growth of adjacent production shelves. For example, temperature probes can be embedded in the substrate, and/or laser probes can be configured inside or outside the growth chamber to monitor mycelium growth. Such data can be integrated within the machine learning model to further guide automated monitoring and production of mycelium.

In an alternative embodiment, the proxy reactor can be utilized without a distinct vessel and may merely be placed (as one or more sensing apparatus) in the pre-existing and actively used bed(s) or tray(s) of a growth environment alongside the growing organism, to understand how novel environmental conditions will affect mycelium growth, adjusting conditions based on the sensed conditions to ensure optimized mycelium growth in a novel environment. The proxy reactor model may also be used to identify particular localized condition fluctuations within a single growth environment and provide AI growth models with data to make condition adjustments caused by localized growth environment fluctuations; adjustments can be either globally or locally implemented within the growth environment to achieve the desired mycelium growth outcome, including in completely novel or recently established growth environments.

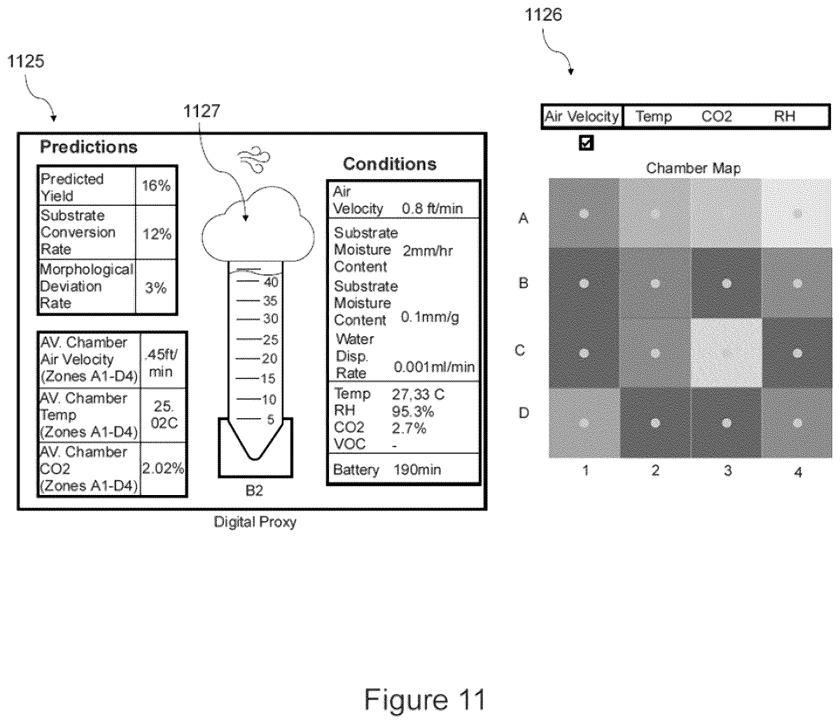

FIG. 11 illustrates an embodiment of conditions and predictions 1125, proxy digital twin reactor 1127 (digital representative of physical growth vessel), and heat map 1126 in the context of a proxy reactor. Such proxy reactor arrangement can lead to AI driven growth environment control. Proxy reactors can, for example, capture the relative humidity of the environment, temperature, CO₂, VOC, airflow velocity, substrate humidity level and the tissue height. This model can be used to dynamically customize the growth environment control settings (of the environmental condition controllers) during a commercial aerial mycelium production (or growth) run. The airflow, relative humidity, and temperature settings can be tuned differently inside a growth environment to minimize environmental condition variation for a given location, and therefore to standardize the overall growth quality within a growth environment bed, rack, or other growth matrix support.

The model can not only capture spatial variation within a large growth environment (e.g., chamber), but also temporal variation within a growth environment (e.g., duration of growth process). The AI associated model with the described hardware can estimate how much aerial mycelium tissue growth is anticipated at a particular time point in the growth timeline (production period) (e.g., day 1=20 mm, day 2=35 mm). Should physical proxy reactors 900 report a deviation in aerial mycelium tissue height from a prediction asserted from the digital twin, the growth environment can automatically compensate for the lack of growth by directing an environmental condition controller to change an environmental condition or introduce a growth accelerant (e.g., nutrient mixed into airflow). The data collected from sensors may be used to train an AI model that can predict how these parameters affect the growth rate of aerial mycelium at different locations within a growth environment. Furthermore, looking at historical data collected from a specific location in a growth environment, the model can estimate if there will be any morphological deviation during aerial mycelium tissue growth at that location and time point. By combining historical data and real-time data, the model can predict the yield of the area that is adjacent to the proxy reactor.

Proxy reactor networks offer a standard measurement and modeling framework that eliminates the need for knowing any proprietary commercial growth environment control infrastructure, should that be a concern of a future aerial mycelium growth partner or concerned third party. The proxy reactors can be deployed to any growth environment and capture the local conditions by growing aerial mycelium in small growth vessels, such as in 910 as illustrated in FIG. 10, and measuring their performance as a proxy to the overall growth/production inside that growth environment. They can report the conditions inside the growth environment and report what needs to be yet achieved for a target yield to be reached. These values can then be converted into environmental set-points and used to drive the growth environment dynamically to achieve the predicted conditions. Proxy reactors 900 can model the conditions of individual aerial mycelium growth/production environments, and the models obtained from previously monitored growth environments can be combined to estimate the production capacity and/or needs of new growth environments. Advantageously, new users can be informed about the theoretical production potential of their growth environment(s) and contribute to the selection of appropriate modeling to maximize efficiency of resources and production for their particular growth environment(s).

This proxy reactor framework can combine historical and real-time data from multiple growth environments, and the reactor networks inside them. Different production cycles that occur across similar growth environments, environmental conditions, species, and substrates/growth matrices can be used to inform each other. The AI-driven framework can then consider data from multiple reactor networks and generate a general model to make predictions that can take into account variations experienced in many local conditions across growth environments globally, resulting in significant cost savings to potential aerial mycelium growers.

In further embodiments, proxy reactors may include peripherals to manipulate aerial mycelium tissue growth via mechanical means or via directed air pressure, to alter the density of aerial mycelium hyphae fiber growth. For example, aerial mycelium tissue growing in a growth vessel such as in 910 as illustrated in FIG. 10, can be pressed down periodically with an air nozzle and the aerial mycelium tissue response can be observed over time. The 3D imaging system can measure the changes in aerial mycelium density and tissue height and report them in real-time to an AI model that can learn optimal rates of intervention for different environmental factors. This system can be used to model different growth substrate (growth matrices) recipes, environmental conditions, and aerial mycelium tissue manipulation methods for different aerial mycelium products. Based on the modeling and simulation data gathered via the proxy reactors 900, large scale robotic intervention systems to alter growth environment bed or tray-size mycelium panels can also be implemented.

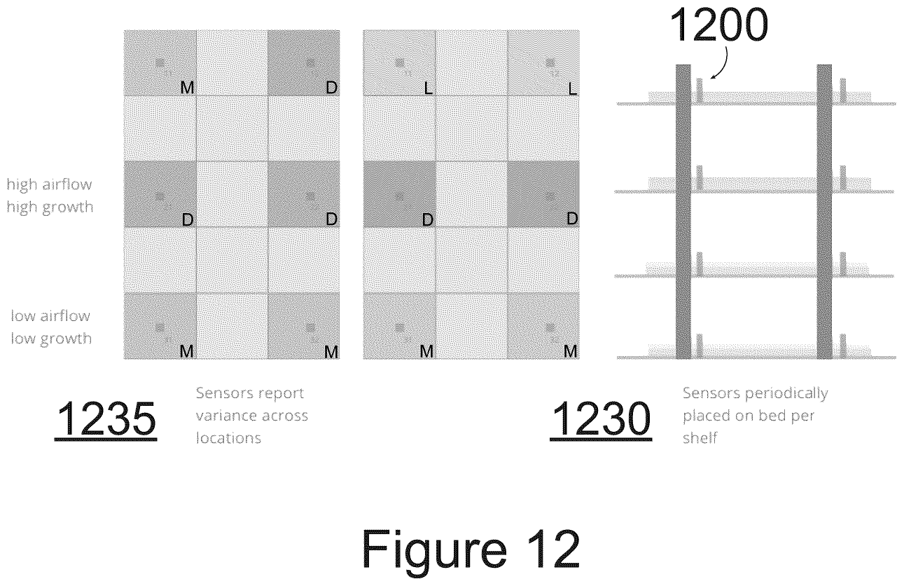

FIG. 12 illustrates one example network embodiment 1230 in which multiple proxy reactors 1200 are placed on racks and shelves in a growth environment. Sensors on the proxy reactors can then report variance across multiple locations 1235 in the growth environment. In the figure, the various placements are distinguished by color gradation or the following indicators: L-light gradation, M-medium gradation, or D-dark gradation.

### Further Details on a Goal-Driven AI Model

Most indoor farming paradigms rely on controlling environmental growth conditions in growth environments. Growth systems often maintain pre-set chamber conditions based on feedback loops. For example, the airflow, CO₂, RH, and temperature can be controlled with direct-drive, reactive, or model-based control paradigms. Pre-set conditions can also be estimated by predictive models which can yield optimal settings to improve yield, growth performance, or minimize energy costs, or other criteria to optimize.

Aerial mycelium-based biomanufacturing has a unique condition in that it is possible to provide a goal or objective that is closer in character to the desired mature mycelium or mycelium-based material produced. For instance, in cotton-based textiles, one cannot train an AI model of growth based on the end product (e.g., yarn, textile, sweater), because when the cotton is grown, it passes through a destructive process (e.g., being spun) and is rebuilt into yarn to be able to view a final textile. Similarly, tomato growers cannot show tomato sauce as an end product, in order to train an algorithm to grow tomatoes, as the tomato is taken through a destructive process in order to produce the tomato sauce.

With mycelium, however, the situation is somewhat different. It has been determined that one can train an algorithm for morphological and mechanical target features that are closer to a desired mature aerial mycelium, with such features optionally incorporated into a mycelium-based product. Even though the mycelium-based products can often be achieved with some additional changes, such as being seasoned in the context of a mycelium-based food product, what is grown throughout the production process has mechanical, structural, and morphological properties that can be parametrically tuned during the growth process that directly influence the desired properties of the mycelium-based product.

This flexibility allows for the provision of many exemplary still images and/or videos of growth processes to train an AI model that can do high-level planning to achieve a desired goal. The goals can be specified with a bitmap pattern or mask, which show fiber orientations on a surface (e.g., masks) or be a raster or vector image that shows a figure, texture, logo and so on.

In such a goal-driven manufacturing paradigm, the algorithm can determine the steps to achieve the goal, the algorithm can break-down the steps or (sub-goals) into discrete tasks to achieve each step and the algorithm can break the tasks into features or parameters that need to be specified to achieve that task. As goals get broken into smaller sub-goals, the algorithms gain more agency in deciding which parameters to select and “black box” the process. As the process can include stage-gates (e.g., goal of day 1 is to grow a certain height and orientation), the manufacturing progress can be transparent to human users, while they can be spared from the underlying complexity of individual decisions to achieve them.

These sub-decisions can be adjusted dynamically in real-time to achieve the goals. Therefore, any unforeseen condition such as contamination or deviant morphology can be addressed quickly and in a customized way. The algorithm may decide to cut out an area to terminate its growth for instance.

A goal-driven system can also operate better in noisy or unstable environments. Instead of assuming a predictable environment, or trying to correct adverse environmental conditions, which are certainly possible and contemplated by certain AI models described herein, the goal-driven algorithm can focus on achieving the desired result given the constraints of the growth environment.

Some additional advantages of a goal-driven AI model for growing aerial mycelium include the following: there could be hundreds of decisions that can be automated and this will provide an order of magnitude more control over the process than humans can perform based on a direct-drive (set and forget) or reactive system (feedback loop); the goal-driven approach does not rely on representing the sub-goals accurately as they can be noisy, inaccurate or incomplete; what algorithms can do well is to compensate them and adjust them as they see fit. In a reactive system, on the other hand, the system may be required to maintain setpoints, but a goal-driven system has the agency to attempt different set-points until a goal is reached; humans will provide the higher objectives in the form of images of desired mycelium or videos that show what needs to be achieved at a given time-point in the process; after hundreds of examples (usually after 40), the algorithms “learn” what features to pick, how to plan and achieve their targets and will use reinforcement learning to get feedback on their performance. For instance, the algorithms will have a cost or error correction function that needs to be minimized to achieve the goal. At different time-points they compare the current production to the target and if they see a deviation, they can re-adjust the parameters, or sub-goals to correct their action.

The goal-driven approach relies on models of growth environments, tissue, substrate, and so on, but they can be basic models that become more detailed over time. This approach can combine top-down (previously learned models) and bottom-up (learning algorithms)

Deep learning models often identify on their own what feature matters to learn to update their models so humans do not need to make decisions about such features. In order to enhance the likelihood of attaining the desired product, humans merely need to get better in showing the end-products or capturing process videos to train the models, which can be accomplished by the systems described herein.

Therefore, in a further example embodiment in accordance with the invention, a goal-driven machine learning platform that can oversee the generation of many models to drive decisions based on qualitative or quantitative objectives is proposed. Rather than humans dialing in setpoints, the machine learning platform dynamically generates or discovers such control set-points on its own.

In additional embodiments of goal-driven machine learning model platforms, an aerial mycelium growth platform can be given a target aerial mycelium morphology, texture, or mechanical performance criteria as an end-goal. A goal-driven model then breaks the goal into subgoals, tasks, and parameters, and decides which model to utilize among alternative machine learning, planning, and prediction paradigms. For instance, to be able to grow aerial mycelium tissue for a specific morphology (e.g., pattern, texture, density, tensile strength, taste and so on (assuming that such attributes have previously been correlated with visual image data), it can pick models from model libraries that allow it to build a set of sub-goal trajectories to achieve the goal. Once the goal is broken into sub-goals, it can utilize specific strain, substrate conversion, growth kinetics, environmental condition models to generate tasks, conditions and parameters to achieve them.

This platform can generate a sequence of sub-goal trajectories and action plans that can be tested. It can run a number of computer simulations to decide which plan would yield an optimal condition. For example, it can run alternative thermodynamic conditions for a growth environment and pick a temp, CO₂ or RH settings. Similarly, the machine learning platform can take into account other environmental growth conditions such as substrate availability, product post-production logistics, or seasonal climatic variables to optimize its output trajectory. These simulations can also be repeated for globally diverse farms or factories with different growth environments, to be able to decide which would be better suited for a specific aerial mycelium production run.

For novel aerial mycelium product applications or when there are gaps in the knowledge base of plans or models, due to sparse data, the platform can design physical experiments as research runs to evaluate hypotheses or find optimal solutions within its solution space.

In still further embodiments, the platform can utilize a policy-based plan execution paradigm to make decisions based on historical aerial mycelium growth runs. This historical data might be obtained from a distributed network of global growth environments.

This model learning and execution paradigm can learn from multiple types of models or the same models obtained at different locations. It can also employ hierarchical methods, where the higher-level goals (e.g. target yield, achieving a specific texture growth pattern, meeting a critical-to-quality criteria, and so on) are divided into smaller goals which may demand their own model learning and execution tasks.

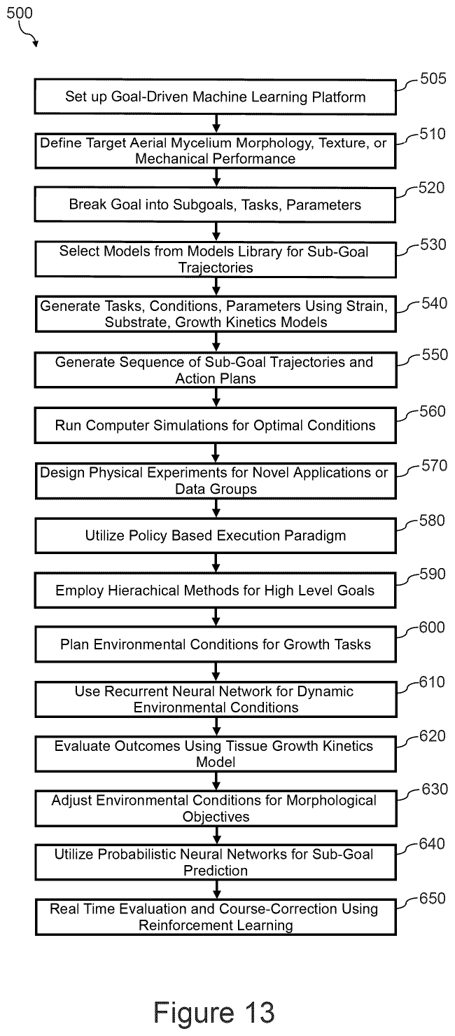

FIG. 13 illustrates an embodiment of a goal-driven model showing a series of steps in diagrammatic fashion. In FIG. 13 a system/method utilizing machine learning models to effectuate desired growth of aerial mycelium 500, includes a goal-driven machine learning platform organized 505 for selectively growing an aerial mycelium having a desired morphology or demonstrated set of physical attributes or both. To reiterate, by “goal-driven” it is meant that a specific pre-defined aerial mycelium material demonstrating a particular set of physical or aesthetic attributes is programmed into the processor and associated model, and based on said selected goal aerial mycelium, the associated model(s) directs the growth environment to create appropriate environment growth conditions that will result in the targeted aerial mycelium (having the desired morphology or physical attributes) being grown. Therefore, a pre-defined target aerial mycelium morphology and/or set of physical attributes (such as for example, those attributes associated with a visual image of mycelium at a particular time within a growth period) is first established 510. Such pre-defined target may include attributes such as for example, morphology (e.g. aesthetic appearance, texture, color, mechanical performance (such as for example, performance based on prior correlation between desired mechanical properties or taste, and specific visual images). Such goal-driven objectives may be optionally broken down into subgoals, tasks, and parameters, such as for example, at various time periods during an aerial mycelium growth timeline 520. If such subgoals are desired to be pursued, select models may be taken from various models in a model library for sub-goal targets or growth trajectories 530. Such subgoal models may themselves be designed to generate specific tasks, conditions, and/or parameters using discrete fungal strains, substrate formulations, and/or growth kinetics models 540. The overall goal-driven model will then arrive at a generated sequence of sub-goal growth trajectories and action plans (if sub-goals are implemented) 550. In one embodiment, it is desirable for the system to run computer simulations at every logical opportunity, for the identification of optimal growth conditions throughout the growth timeline, and reduction of heretofore unforeseen environmental conditions risks 560. Concurrently, physical experiments may be designed and implemented for additional novel products and/or data gaps, which can then be incorporated into associated data management systems 570. As part of the system-model(s), the system may utilize a policy-based plan execution paradigm 580. The system may employ hierarchical methods for achievement of high-level goals 590. Additionally, the system can assist in planning environmental conditions for growth tasks 600.

The system may utilize recurrent neural network(s) for dynamic environmental condition monitoring and adjustments 610. Following such aerial mycelium growth, the grown material may be evaluated using tissue growth kinetics modeling 620. At the appropriate time in the mycelium growth cycle, environmental conditions may be adjusted by environmental condition controllers for predefined morphological or other physical attribute objectives (target goals) 630. The system optionally contemplates further, that should priorities for target materials change during the growth timeline, and sufficient time remains to allow for alteration of conditions correlated to differing material attribute target goals, the system may be sufficiently robust to implement a course correction with modified conditions, upon notification through human intervention, to pursue the alternative priority target material course. The system may also utilize probabilistic neural networks for sub-goal prediction 640 as appropriate. During the entire growth timeline, the system may conduct real-time evaluation and course-correction using reinforcement learning 650.

### Research Model and Design of Experiments Model

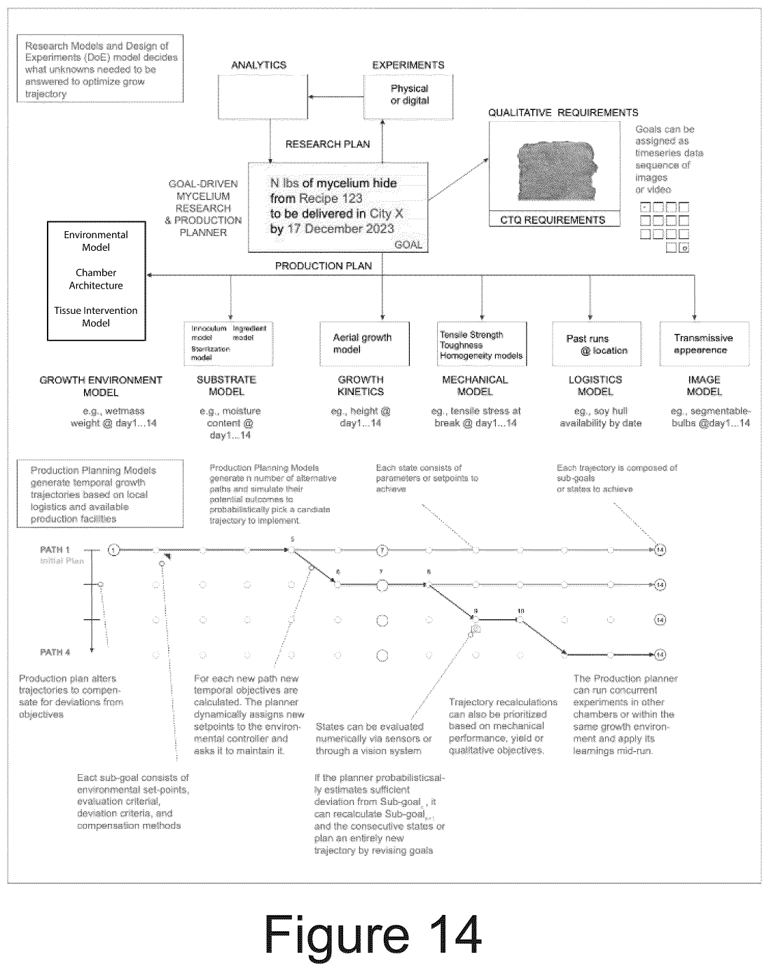

FIG. 14 illustrates an embodiment of a goal-driven mycelium research and production planning platform that can utilize a multi-modal architecture to initiate and execute decisions for a mycelium production run. In a specific production run, the temporal and spatial parameters of a growth environment can be driven by a high-level planning algorithm that can break down the needs of a growth task into smaller steps. For each rack, shelf, bay position or other growth supporting matrix, the proposed AI model can generate environmental conditions accordingly and realize them with specific actuators such as fans, mechanical tissue manipulators and so forth. The planner can drive the growth environment dynamically through environmental growth conditions based on a model that includes specific thermodynamic that can predict sequential or evolving interactions between airflow, RH, temperature, CO₂, and so on using a recurrent neural network (e.g., long short-term network).

The “research planner” component of the platform involves a “design of experiments (DoE)” and experiment “analytics” evaluation modules. The “Experiments” module can initiate, plan, and execute both digital and physical experiments inside the growth chamber. Digital research experiments may involve simulations or predictions based on existing models given a new goal. Physical research experiments may involve running specific growth runs in small form factor vessels (e.g., petri dishes, tubes, trays) to validate hypotheses or provide more information on a specific aspect of the goal that may not be inferred from an existing model. The physical research experiments can run in parallel during a production run and can have accelerated execution timelines that are different from an actual production run. The “analytics” module can provide real-time statistical or probabilistic evaluation of the experiments and provide information for the later stage goals of a production run. The “production planner” component of the platform utilizes a number of models and nested models to generate a “production path” to achieve the growth goals. These models include but are not limited to environmental conditions models, substrate model, growth kinetics model, mechanical performance model, logistics, and imaging-based evaluation models. Depending on their domain, these models describe relationships between input parameters and output responses. For example, the growth kinetics model may include a neural network that describes the relationship between growth parameters (e.g., branching factor) and series of responses (e.g., tissue density, growth rate). A production path is selected after evaluating a number of possible paths based on different constraints and affordances informed by substrate, chamber conditions, or logistical needs at different locations. Production paths consist of a series of sub-goals that need to be achieved to accomplish the main goal. As the path gets executed, the planner monitors the state of the plan and adjusts it to minimize deviations. It uses real-time sensors to monitor how close the current product is close to the intended sub-goal and changes the plan trajectory as needed. To be able to evaluate the outcomes of the environmental conditions at a given time, it can utilize the tissue growth kinetics model, which can predict the growth rate (e.g., aerial mycelium growth in mm per second), branching factor, and so on to decide state-based sub goal conditions to achieve over the course of the growth period. The process of re-calculating sub-goals and changes in the path trajectory involves re-calculating set-points, changing frequency of actuations (e.g., mist deposition duty cycle), and rate of physical interventions (e.g., airflow activation at specific locations) and so on.

The platform can have different steps or states such that it can gauge the outcome of its current progress. To be able to evaluate the outcomes of the environmental conditions at a given time, it can utilize a tissue growth kinetics model, which can predict the growth rate (e.g., aerial mycelium growth in mm per second), branching factor, and so on to decide state-based sub goal conditions to achieve over the course of the growth period. The environmental growth conditions can be tuned dynamically to achieve a specific tissue height, density, or morphology as a set of temporal objectives to be met by a given time (e.g., state 1: arrive 10 cm by day 1, state 2: arrive 10 cm by day 1).

To achieve the morphological objectives of the given states, it can utilize a substrate model that utilizes substrate conversion rates, yield, and waste calculations in relation to growth speed and so on. The morphological objectives can be predicted based on the outcomes of probabilistic neural networks (PNN) that can set qualitative or quantitative sub-goals and a probabilistic distribution of how it can be achieved given the uncertainties caused by real-time conditions, interruptions and noise.

The performance of the high-level planning algorithm's goal achievement timeline and task-execution trajectories can be numerically evaluated in real-time using reinforcement learning (e.g., cost or error function). In this fashion, the platform can course-correct or build new trajectories that can minimize deviations or maximize profit if it encounters an adverse condition. For example, if during a production run, regions with poor growth are observed, the algorithms can start a process for calculating additional income from the material if channeled into alternative revenue streams such as colonized mushroom substrate for biogas production.

### AI Modeling Mycelium Growth Process as Data Animations

In still a further example embodiment of the invention mycelium growth parameters may be captured independently of the growth environment and control paradigm. A growth environment equipped with location-specific image systems (e.g., still or video image sensors) can use aerial mycelium as its own response variable. Instead of creating a model for the environmental conditions, the model can utilize “data-animations” that describe aerial mycelium growth as a sequence of conditions, objectives, states, or parameters captured like the key frames or states of an animation.

In such an embodiment, mycelium data animations (MDA) are a series of data frames that describe aerial mycelium morphology/appearance and growth rate. Similar to video animations, the data frames capture an overall process sequentially, frame-by-frame, or step-by-step. But in addition to visual information, the data frames can include other types of data. The information in these frames can be both about changing and constant information. Changing information would include growth rate in the form of ratio of black and white pixels in a detection window and_mist velocity (liquid mist particles in the air). Non-changing information would include temperature, CO₂, and RH, values that are fixed within a recipe.

MDAs provide a concise way of representing all information needed to bring the growth of mycelium from frame 1 to frame n. On a temporal scale, these frames will describe how aerial mycelium tissue morphology needs to look from day 1, until the end of the growth timeline (aka growth cycle).

MDAs provide an algorithm with a grow-room independent, mycelium-focused representation. Instead of tuning environmental growth conditions to maintain a specific atmospheric condition, a growth environment can be driven in a reactive mode to maintain the goals set by the data frames of aerial mycelium growth.

An important advantage of MDAs is that they don't have to be based on actual growth runs inside physical growth environments. The data frames and their animations can be generated via algorithms, which can generate many MDAs based on different environmental growth conditions and allow a deep learning network to learn how to grow it, as deep learning networks are particularly effective at learning from examples. They can generate similar results based on very short fragments of audio and video clips. In one example, a deep learning network would be trained with a relatively large sum of MDAs that allow it to understand what parameters need to be maintained and which need to be changed to progress aerial mycelium growth from a tissue height of 0 cm to 10 cm. The data clips and animations can be generated using diffusion-reaction algorithms that can generate dozens of possible growth outcomes that vary yield, growth morphology, texture, pattern, and so on.

It is envisioned that MDAs will be available with a key that translates how the different frames can be achieved using growth environmental controls. These keys can map out what growth environment control settings (e.g., mister, CO₂ tank release valve, heating/cooling coil, airflow directing agents) need to be altered to achieve the higher and lower end of the values needed in the data frame. For instance, if a data frame describes 20 units of change in airflow velocity between frame 1 and 2, the algorithm opens and closes the airflow directing agents accordingly to meet that need. The chamber control algorithm would not need to understand anything beyond the next keyframe and can follow a Markov-chain-like process. The temporal distance (duration) between each frame can also be specified at different intervals. So, the amount of time it would take to arrive from Frame 1 to Frame 2 can be 10 minutes, whereas the time taken to arrive from Frame 2 to Frame 3 can be 20 minutes or another duration, and so on. By providing different keys to different growth environment control paradigms, MDA-based learning algorithms can realize any MDA in any growth environment without necessarily trying to model a complex growth environment with dozens of input variables.

A minimum viable MDA can be described with: 1) ratio of black pixels (background) to white pixels (aerial mycelium) within an image or a region of an image captured by a 2D image sensors; 2) ratio of black pixels (background) to white pixels (liquid mist particles in the air) captured by a 3D image sensors. The invariant information, or what needs to be kept constant (e.g., RH, CO₂, and temperature) can be recorded mainly to capture deviations. Similar to the practice of animation, only the changes in the scenes/frames would be captured in the keyframes.

The image systems (e.g., image sensors) for such embodiment may be mounted on proxy reactors (as illustrated in FIG. 10) or directly embedded onto beds, trays, or other growth matrix supports of the growth environment. MDA captured with different image sensors systems can show variability in resolution, exposure, depth of field, lens angle and so on. To be able to translate the visual growth information (e.g., ratio of white/black pixels that indicate growth rate) across different image sensors systems, machine vision paradigms such as “template matching”, scale invariant feature transform (SIFT) or oriented FAST and rBRIEF (ORB) may be used. These algorithms can scale, translate, and perspective map the visual frames so that MDAs captured with one system can be utilized by another.

Unlike generalized aerial mycelium growth models that may be more challenging to reproduce, MDAs may be seen as possible growth trajectories that can be verified digitally or through actual runs to build degrees of confidence. Unlike other models, MDAs do not provide linear or differential relations between parameters, but rather a sequence of data frames that depict instances of growth between time frames.

### Air, Aerial Mycelium Fiber, and Substrate Voxels; In-Situ Manipulation and Testing of Air, Aerial Mycelium Fibers, and Substrate During Aerial Mycelium Production Runs

Design-prototype-manufacture of aerial mycelium products may present a lag between mechanical testing and experimental design. Biopolymer tissue grown under different substrate, strain, and environmental conditions are often evaluated at the end of the growth cycle (aka growth timeline) for their mechanical performance (e.g., tensile strength) or appearance (e.g., homogeneity, texture, uniformity, regularity) to inform the new set of experiments or manufacture runs.

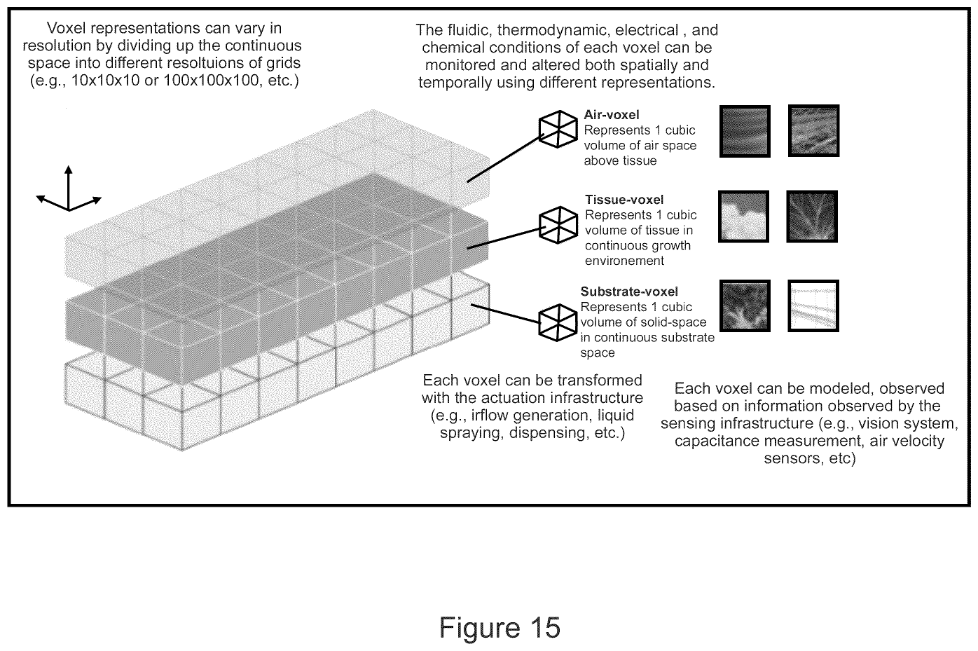

FIG. 15 illustrates an embodiment of an alternative modeling paradigm. A number of key interventions into the growth environment that utilize this paradigm occur, for the in-situ analysis and manipulation of air, fibers and substrate, so that the candidate treatments can be observed, tested, and manipulated during a growth run.

### Air-Voxels and Robotic Airflow Generators

The airspace above the growth environment growth surface is divided into a grid of air-voxels (3D representation of volume as cubes), in which air velocity and mist activity can be monitored in real-time with an image system. In one embodiment, 3D stereoscopic infrared image sensors can be used to measure the mist-in-the air velocity and direction. The air-voxels discretize the continuous growth space for aerial mycelium and are used to model the distribution of flow activity above the tissue. The airspace discretization allows digital modeling of the space as a 3D matrix of addressable locations where flow activity and the boundary conditions of fluidic behavior can be modeled relationally.

Each air voxel can be manipulated with a robotic airflow generator that can provide a custom airflow intensity to the voxel location. The airflow generators can be mounted on the rails or side structures of a bed or other growth matrix supporting structure, which run parallel to the sides of the bed or other structure that is above or at the same height. These generators may have 360-rotating servo-controlled fans that can direct the airflow into desired voxels or clusters of voxels. Airflow can also be introduced via a fleet of drones that can fly over the growth surface and hover above specific voxel locations to provide specific airflow conditions such as directed laminar flow or turbulent air.

### Fiber-Voxels and In-Situ Fiber Puller and Pushers

Tissue is grown in a bed or other growth matrix (of at least substrate) supporting structure, broken down into a matrix of sub-regions (fiber-voxels), which can model the growth activity above the substrate. Each fiber-voxel discretizes the continuous growth area and represents a volume of tissue that will grow in a defined location (x, y, z). Fiber-voxels can be represented by an imaging system that is equipped with 2D, 3D, infrared, and thermal image sensors. The tissue space discretization allows digital modeling of the space as a 3D matrix of addressable locations where activity e.g., fiber formation, branching, merging, expansion, and so on can be modeled. Each fiber-voxel can be observed as a distinct footprint. Multiple fiber-voxels can be tracked over time to understand relational behavior.

At each fiber-voxel location, aerial or other mycelial tissue can be grown on special scaffolds suspended from the roof of the shelf above, via fishing lines or other connectors. Fiber Pullers may be released over the surface of the bed during growth and slowly roll up with a pulley mechanism. The tension on the fishing line or other connector is periodically measured with a spring gauge or a line tension gauge. As aerial or other mycelium tissue grows over the scaffold, the pull of the fishing line will start to detect a measurable pull caused by aerial or other mycelium. As different parts of the bed can be sampled with this method, it becomes possible to characterize the tensile strength of wet tissue at different stages of its growth (e.g., day 1, 2, 3). This information can be utilized through a growth environment control feedback loop that can alter environmental growth conditions (e.g., reduce airflow or mist deposition) to make the tissue growth denser or lighter. The information can also be utilized by a goal-driven AI algorithm which can compare the results from different regions and customize the mist and airflow distribution to the regions that need more or less treatment such as rotating bed platform directions or mobilizing robotic airflow generators.

Fiber pullers can also be used to press down aerial mycelium tissue by releasing the pulley mechanism back onto the tissue and also function as an in-situ fiber intervention tool. The tissue's response to periodic press-down and pull-up analysis can be done across a production run. The information sourced from sampled sections can be utilized to drive the condition of the airflow of neighboring zones. All three components can be used together to sample, analyze, and alter growth conditions during a run, merging the mechanical analysis of tissue with its growth manipulation.

### Linear Platform for Manipulation of Growing Mycelium Fibers

Current aerial mycelium growth in beds relies on controlling or manipulating the entire growth environment of essentially a large 3D box. While this can be done with success, it cannot be done as precisely as one would desire (say within a 10×10×10 cm space). This better precision would be desirable to influence the growth of aerial mycelium which grows on an order of 0.01×0.01×0.01 mm and also over the temporal domain (t=minutes)

In order to address this need, a local environment optimization, built on the same chassis used by mushroom farms for harvesting mushrooms and otherwise manipulating mushroom beds is now proposed. The core operation relies on an existing tuned aerial mycelium growth environment with a target set of generalized conditions which produce aerial mycelium. This operation is then improved by a machine that runs upon two bed or other support structure side rails or other side support structures of each bed in a linear fashion.

As the machine moves up and down a bed or other growth matrix supporting structure (time period can range from 10 seconds to 10 hours to traverse the length of a bed) it uses various “actuator heads” to manipulate the environment and in some cases, the aerial mycelium growing directly beneath it. In this fashion, one is able to more precisely control the local environment of growing aerial mycelium, and dynamically change it over time.

In one example embodiment, the bed runner has an array of 100 1×1 cm fans pointed at a 45-degree angle to the growing mycelium. Each fan is controlled by a computer controller which can vary the rpm from 0-9,0000 RPM. The fast runner travels up and down the bed and using feedback from an image system (image sensors) mounted on the fast runner, compares the surface condition of the aerial mycelium on the bed to the target surface condition. Using the computer fans, it dynamically changes the airflow rate directly above the bed. This either adjusts the bulk environment above the bed, or may be used to directly manipulate the growing aerial mycelium fibers, matting them together, weaving them into strands, or otherwise forcing them to touch and agglomerate (or separate in some cases)

This same platform (which moves spatially during a run, like a “print head”, leaving traces of action of the entire period) may be adopted with other print heads, which can deposit materials, fats, solids, or living organisms. They may include fibers or rods or pins that are pushed, pulled, or otherwise impacting the surface, as well as other non-invasive tools such as electron beams, lasers, lights, and sensors such as VOC's sniffers, for localized sampling.

### Substrate-Voxels (or Growth Matrix Voxels) and In-Situ Manipulation of Substrate Conditions

Substrate-voxels discretize the solid space below aerial mycelium tissue that may include substrate, moisture, gases-among solid particles, inoculum, substrate-colonizing-mycelium, growth-conditioning structures, and other solid media. The substrate voxels provide a temporal and spatial representation of the interior space and can be used to actively monitor and manipulate mycelial activity. Through the substrate-voxels, it is possible to characterize electrical, chemical, and biological changes in the solid space due to mycelial colonization activity. They provide metrics to quantify the rate of mycelium colonization with regards to PH change; metabolic activity via charge and capacitance distribution, and CO₂ production and so on.

Each substrate voxel can be equipped with growth conditioning structures that can manipulate the tectonic activity inside the space. For example, different geometric blocks can be placed inside the voxels and provide direction or constraints to guide mycelial activity into different growth behavior. These spatial interventions can change the rate of growth, length of mycelial networks, as well as distribution of mycelial fibers that can arrive to the surface of the substrate to initiate aerial growth.

In addition to the passive structures, the substrate voxels can be actively manipulated to provide nutrients, moisture, and other solutes through pipes and embedded liquid dispensing systems. For example, the moisture content of each voxel can be tuned dynamically by sourcing different amounts of liquids in a given region.

Air-, tissue-, and substrate-voxels can be used together to build a more comprehensive model of aerial mycelial activity as it extends from the solid substrate to aerial tissue. Voxels can be optimized in relation to each other both spatially and temporally. By monitoring the different stages of voxel behavior, a machine learning model can provide predictions to optimize growth conditions and inform different in-situ activation methods at different stages of substrate colonization and aerial growth.

Each voxel model can also provide states for mycelial data animations (e.g., target tissue density at voxel footprint) and goals for the goal-oriented aerial mycelium planning and execution algorithm (e.g., bitmap voxel growth pattern as morphology or texture).

### Substrate-Directed AI Modeling; Initial Substrate Selection Model; Locally Available Substrate Selection Model

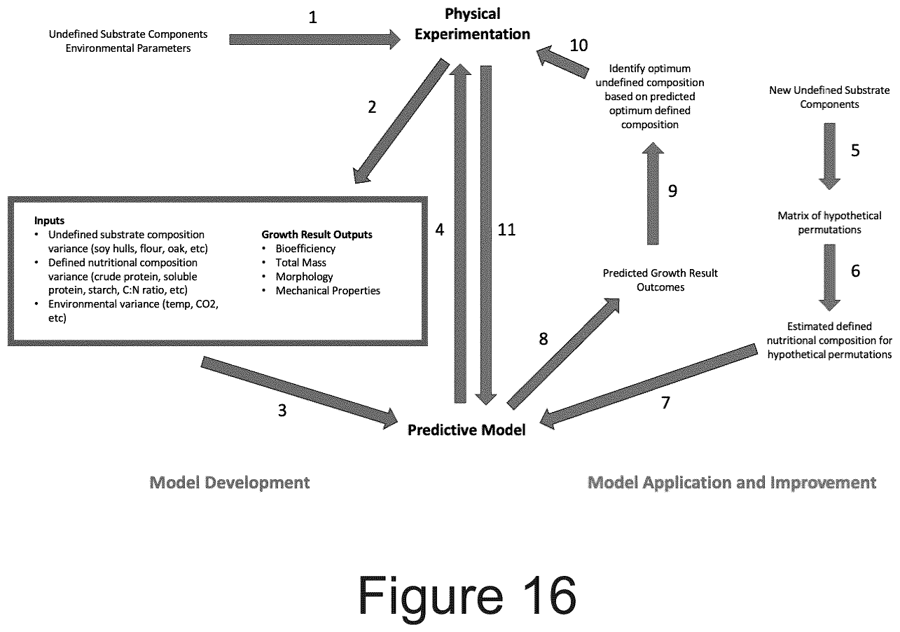

FIG. 16 illustrates an embodiment of a system for using AI models to select for or define substrate suitable for growing aerial mycelium. A more detailed example of the method illustrated in FIG. 16 includes the following specific steps. The following detailed method is provided as a single, non-limiting example of substrate-directed AI model.

### Example of Substrate-Directed AI Model:

1. Ranges of interest for substrate component loadings and environmental variation are passed to physical experimentation.
2. Data from physical experimentation is generated, correlating all inputs; environmental variance, undefined substrate composition variance, and defined nutritional composition variance where the defined nutritional profile is a function of the undefined substrate composition.
3. A predictive model is trained on all data optimizing prediction of growth outputs as a function of undefined substrate, defined nutrition, and environmental inputs.
4. Optimum experimental permutations are generated to further improve the model by prioritizing experimental permutations with the greatest prediction uncertainty. Steps 1-4 are repeated as a loop until model quality reaches a minimum defined level for practical application.
5. New undefined substrate components are identified based on geographical, economic, and/or technical limitations. A matrix of hypothetical permutations of these identified substrate components are envisioned based on economic and technical limitations.
6. Defined nutritional composition for each permutation in the matrix are estimated based on the nutritional composition of the discrete substrate components.
7. The matrix of defined nutritional compositions for each hypothetical permutation of undefined substrate compositions, along with environmental assumptions, are tested within the predictive model.
8. Predicted growth results for each tested hypothetical permutation are output.
9. Predicted growth results are evaluated for hypothetical permutations that demonstrate probable optimal growth.
10. Selected hypothetical permutations with the best probable growth responses are passed to physical experimentation in order to validate growth response.
11. Results from physical experimentation (correlating defined nutritional composition, undefined substrate composition, and growth response) are added to the predictive model in order to improve the model's quality for making predictions for the newly identified undefined substrate components.

The various illustrative logics, logical blocks, modules, circuits and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general-purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, e.g., one or more modules of computer program instructions, encoded on a computer storage media for execution by, or to control the operation of, data processing apparatus. If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable media includes both computer storage media and communication media including any medium that can be enabled to transfer a computer program from one place to another. A storage media may be any available media that may be accessed by a computer.

Additionally, the operations of a method or algorithm may reside as one or any combination or set of codes and instructions on a machine readable medium and computer-readable medium, which may be incorporated into a computer program product.

Therefore, the described invention presents an analytical method (using a machine learning tool) for determining either irregular environmental growth conditions in a mycelium growth environment or alternatively, the presence or absence of select visual details/physical attributes in growing or grown aerial mycelium, for the purposes of quality control, condition adjustment, or final material selection (from a range of predefined physical attributes) that may be observed from growing aerial mycelium. Alternatively, the analytical method may accomplish both objectives of determining irregular environmental growth conditions and adjusting them accordingly and directing change of said environmental growth conditions based on predefined but observed physical attributes of the growing or grown aerial mycelium. Such invention may be applied to each type of fermentation process which is designed to grow aerial mycelium. The invention contemplates a feedback loop control system of mycelium growth using a “diffusion” type trained model system, in that the model would be initially trained on physical attributes of aerial mycelium and environmental conditions which impact such physical attributes. Such model would then control growth within a mycelium growth environment (e.g., a growth chamber or a bioreactor). In this fashion, there would be a continuous feedback control loop in operation within a mycelium growth environment or select or all important growth parameters (such as air gases, air flow, mist levels, mist liquid composition, mist direction, temperature, relative humidity, light levels, and nutrition and such), based on a visual target that is previously established and continuously monitored and compared with historical data, where appropriate).

In an alternative embodiment, the system has been taught (through AI modeling) the conditions necessary to produce a desired mycelium product (and which are associated with historical images of successfully growing or mature mycelium products), and will operate independently of human interaction, once the image(s) itself of the desired mycelium is programmed into the system. The system will then dynamically adjust conditions as appropriate to reach the desired mycelium, despite differences in growth chambers equipment and available technology.

In example embodiments, the use of the Persona-Malik model for stable diffusion of images, in combination with a supervised learning approach for training the model, offers a unique approach for optimizing growth of an organism that would otherwise present commercial scale challenges. The use of a computer image system (image sensors with processor as described), to continuously analyze a video feed and adjust environmental conditions through environmental controls offers a more precise tool to assure desired outcomes for aerial mycelial growth. While in the past, individual environmental conditions have been adjusted (e.g., static controls) during the growth of aerial mycelium, an active, real time, responsive approach presents a more targeted growth paradigm for aerial mycelium, which is more likely to allow for large scale production of aerial mycelium based on different organism strains, different mycelium growth environments (rooms, hardware, chambers etc.), and different growth physical locations.

In example embodiments, use of machine learning tools to select appropriately matched flora to provide previously identified nutritional needs for specific fungal organisms (through selected, locally available substrates), will allow for efficient and cost-effective commercial production of aerial mycelium materials, despite geographic differences. In a further example embodiment, undefined substrate (e.g., food waste, recycled lignocellulosic or other materials, or any other upcycled or recycled nutritive source) can be analyzed by the machine learning tool to adapt the growth environment and/or environmental conditions to account for any deficits or surpluses in the nutritional requirements of the growing mycelium caused by the undefined attributes of the a substrate. For example, food waste can be used as a substrate, wherein the amounts and types of nutrients vary. Each delivery of food waste can be analyzed by the machine learning tool to determine nutritional deficits and/or surpluses and adjust the environmental conditions and growth environment configurations accordingly.

As such machine learning tools/modeling may assist in the efficient allocation of resources and the adjustment of conditions within a single growth environment, such as to accommodate disparate conditions in a single environment, machine learning tools/modeling may therefore assist in the efficient allocation of resources or environmental conditions in the different regions to effectuate the desired aerial mycelium material designs and production steps. Other arrangements are also contemplated to be in the scope of this disclosure.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein. Additionally, a person having ordinary skill in the art will readily appreciate that the terms “upper” and “lower” are sometimes used for ease of describing the figures and indicate relative positions corresponding to the orientation of the figure on a properly oriented page and may not reflect the proper orientation of a feature as implemented.

### Scope of Disclosure

It will be understood that although the present disclosure is discussed within the context of food and textiles, the embodiments described herein can be implemented in food, non-food, or other non-textile applications. Additionally, the following non-exhaustive list provides additional fungal genera and species which may be implemented, if not otherwise inconsistent with the present disclosure:

### Implementations

In some aspects, the present disclosure provides for an aerial mycelium, and for methods of making an aerial mycelium, wherein the aerial mycelium is a growth product of a fungus. In some embodiments, the fungus is a species of the of the phylum Basidiomycota, Ascomycota or any of the early branching lineages of fungi, formerly referred to as Zygomycota.

The various illustrative logics, logical blocks, modules, circuits and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general-purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, e.g., one or more modules of computer program instructions, encoded on a computer storage media for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable media includes both computer storage media and communication media including any medium that can be enabled to transfer a computer program from one place to another. A storage media may be any available media that may be accessed by a computer.

A software module may reside in random access memory (RAM), flash memory, read only memory (ROM), electrically programmable ROM (EPROM), electrically erasable programmable ROM (EEPROM), registers, hard disk, a removable disk, a CD ROM, or any other form of storage medium known in the art. A storage medium is coupled to the processor such that the processor can read information from, and write information to, the storage medium. In the alternative, the storage medium may be integral to the processor. Disk and disc, as used herein, include compact disc (CD), laser disc, optical disc, digital versatile disc (DVD), floppy disk and blue ray disc where disks usually reproduce data magnetically, while discs reproduce data optically with lasers. Combinations of the above should also be included within the scope of computer readable media. The processor and the storage medium may reside in an ASIC. The ASIC may reside in a user terminal. In the alternative, the processor and the storage medium may reside as discrete components in a user terminal.

Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of a feature as implemented.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

The word “exemplary” is used herein to mean “serving as an example, instance, or illustration.” Any aspect or embodiment described herein as “exemplary” is not necessarily to be construed as preferred or advantageous over other aspects or embodiments. Various aspects of the novel systems, apparatuses, and methods are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of, or combined with, any other aspect described. For example, an apparatus may be implemented, or a method may be practiced using any number of the aspects set forth herein. In addition, the scope of the disclosure is intended to cover such an apparatus or method which is practiced using other structure, functionality, or structure and functionality in addition to or other than the various aspects of the disclosures set forth herein. It should be understood that any aspect disclosed herein may be embodied by one or more elements of a claim.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Thus, as used herein, a phrase referring to “at least one of X, Y, and Z” is intended to cover: X, Y, Z, X and Y, X and Z, Y and Z, and X, Y and Z.

The headings provided herein, if any, are for convenience only and do not necessarily affect the scope or meaning of the devices and methods disclosed herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method for controlling environmental conditions of one or more growth environments for optimizing mycelium growth, the method comprising:
providing one or more growth environments comprising one or more fungal inocula and a substrate;
providing a monitoring system that transmits data to one or more processors, wherein said monitoring system comprises a machine learning tool executing on said one or more processors that monitors at least one of environmental conditions and mycelium growth in said one or more growth environments; and
applying said machine learning tool to determine one or more changes to said environmental conditions based on said data, wherein said data comprises real-time monitoring information on said environmental conditions or said mycelium growth within said one or more growth environments.

2. The method of claim 1, wherein said one or more growth environments include one or more environmental control devices.

3. The method of claim 1, wherein said mycelium is aerial mycelium.

4. The method of claim 2, further comprising, under control of said one or more processors, activating at least one of said one or more environmental control devices to implement said one or more changes to said environmental conditions.

5. The method of claim 2, wherein said one or more environmental control devices comprise at least one of a temperature control device, a mist control device, a relative humidity control device, an atmospheric pressure control device, and an atmospheric gas control device, an airflow control device, said monitoring system analyzing said mycelium growth by image or video sensing of at least one or more of temperature, mist level, mist liquid composition, mist direction, relative humidity, atmospheric pressure, or atmospheric gases, airflow velocity, airflow volume, airflow direction, electromagnetic radiation, visual light, nutritional supplements in said one or more growth environments.

6. The method of claim 1, wherein said machine learning tool has been previously trained to recognize optimum mycelium growth conditions selected from one or more datasets comprising at least one of:
previous predefined successful growth runs of a same fungal species and/or strain;
a selection of preferred accumulated growth conditions for said fungal species and/or strain; and
a selection of growth conditions specific to said fungal species and/or strain based on at least one of: said fungal species and/or strain lifecycle, predefined mycelium growth image or video sensing data for a time period in a mycelium growth cycle, predefined mycelium growth image or video sensing data for a select successful growth run of said same fungal species and/or strain, and/or predefined mycelium growth image or video sensing data for a desired mycelium product outcome.

7. The method of claim 1, wherein said monitoring system comprises an image system, said monitoring system periodically viewing said growing mycelium for either capturing and later transmitting, or capturing and transmitting in real time, continuous data of said growing mycelium to said processor, said processor communicating with said machine learning tool.

8. The method of claim 7, wherein said image system comprises an imaging or video sensor system in said one or more growth environments for capturing and transmitting either periodic still photographic images or a continuous video feed of said growing mycelium to said processor including said machine learning tool.

9. The method of claim 8, wherein said imaging or video sensor system in said one or more growth environments comprises continuous video feed data, said continuous video feed data is preprocessed to remove data noise and enhance data quality.

10. The methods of claim 7, wherein said machine learning tool is applied to said preprocessed data to perform stable diffusion smoothing while preserving features including at least one of edges or corners.

11. The method of claim 7, wherein said machine learning tool is a Perona-Malik trained model.

12. The method of claim 10, wherein said machine learning tool utilizes a supervised learning approach to train a model, said model being trained using said preprocessed data, and teaching said model to recognize different patterns and structures associated with specific growth stages, growth conditions, deviant mycelium morphologies, or desirable physical attributes previously associated with predefined data patterns.

13. The method of claim 11, wherein said trained model is validated by testing said trained model on pre-validated data to assess the accuracy of said trained model in detecting different growth stages, growth conditions, deviant mycelium morphologies or desirable physical attributes previously associated with predefined data patterns.

14. The method of claim 11, wherein said trained model continuously analyzes said video feed data from said image system and subsequently analyzes and adjusts said environmental conditions to promote optimal mycelium growth.

15. The method of claim 11, wherein said trained model is monitored and refined based on ongoing analysis and observed growth outcomes to achieve desired growth results.

16. The method of claim 11, wherein said trained model is trained to consider downstream production factors such as one selected from the group comprising of yield weight, surface topography, flavor-indicating features, presence of color variations, and tensile strength.

17. The method of claim 10, wherein the monitoring system comprises multiple models in evaluating grown mycelium materials.

18. The method of claim 10, further comprising providing supplemental machine learning tool inputs for consideration by said machine learning tool,
wherein said inputs are based on collected growth run data, substrate data or equipment data.

19. The method of claim 1, wherein said one or more growth environments comprise one or more sub-environments with aerial mycelium at different growth stages, and wherein said one or more sub-environments are comprised of said machine learning tool that is trained to observe how different environmental conditions impact aerial mycelium growth progression by monitoring said environmental conditions at various lifecycle stages of said aerial mycelium.

20. The method of claim 1, wherein said one or more growth environments comprise two or more fungal species and/or strains, and said machine learning tool is trained to learn how different growth conditions or different substrates optimize growth of each of the said two or more fungal species and/or strains, how different growth conditions or different substrates optimize growth of said two or more fungal species and/or strains when grown together, and/or how different growth conditions or different substrates in different sub-environments optimize growth of said two or more fungal species and/or strains.

21. The method of claim 1, wherein said machine learning tool is integrated with intranet or internet query communication tools such that clarification information for continuous improvement of observed data from said image system is accessible to said machine learning tool.

22. The method of claim 1, wherein said machine learning tool is taught to implement learnings from prior stored run successes and failures for future runs.
